SEQUENCE LISTING (1 sequence)

SEQ ID NO: 1 - protein, 2177 aa, Homo sapiens
MAAFGILSYEHRPLKRPRLGPPDVYPQDPKQKEDELTALNVKQGFNNQPAVSGDEHGSAKNVSFNPAKISSNFSSIIAEKLRCNTLPDTGRRKPQVNQKDNFWLVTARSQSAINTWFTDLAGTKPLTQLAKKVPIFSKKEEVFGYLAKYTVPVMRAAWLIKMTCAYYAAISETKVKKRHVDPFMEWTQIITKYLWEQLQKMAEYYRPGPAGSGGCGSTIGPLPHDVEVAIRQWDYTEKLAMFMFQDGMLDRHEFLTWVLECFEKIRPGEDELLKLLLPLLLRYSGEFVQSAYLSRRLAYFCTRRLALQLDGVSSHSSHVISAQSTSTLPTTPAPQPPTSSTPSTPFSDLLMCPQHRPLVFGLSCILQTILLCCPSALVWHYSLTDSRIKTGSPLDHLPIAPSNLPMPEGNSAFTQQVRAKLREIEQQIKERGQAVEVRWSFDKCQEATAGFTIGRVLHTLEVLDSHSFERSDFSNSLDSLCNRIFGLGPSKDGHEISSDDDAVVSLLCEWAVSCKRSGRHRAMVVAKLLEKRQAEIEAERCGESEAADEKGSIASGSLSAPSAPIFQDVLLQFLDTQAPMLTDPRSESERVEFFNLVLLFCELIRHDVFSHNMYTCTLISRGDLAFGAPGPRPPSPFDDPADDPEHKEAEGSSSSKLEDPGLSESMDIDPSSSVLFEDMEKPDFSLFSPTMPCEGKGSPSPEKPDVEKEVKPPPKEKIEGTLGVLYDQPRHVQYATHFPIPQEESCSHECNQRLVVLFGVGKQRDDARHAIKKITKDILKVLNRKGTAETDQLAPIVPLNPGDLTFLGGEDGQKRRRNRPEAFPTAEDIFAKFQHLSHYDQHQVTAQVSRNVLEQITSFALGMSYHLPLVQHVQFIFDLMEYSLSISGLIDFAIQLLNELSVVEAELLLKSSDLVGSYTTSLCLCIVAVLRHYHACLILNQDQMAQVFEGLCGVVKHGMNRSDGSSAERCILAYLYDLYTSCSHLKNKFGELFSDFCSKVKNTIYCNVEPSESNMRWAPEFMIDTLENPAAHTFTYTGLGKSLSENPANRYSFVCNALMHVCVGHHDPDRVNDIAILCAELTGYCKSLSAEWLGVLKALCCSSNNGTCGFNDLLCNVDVSDLSFHDSLATFVAILIARQCLLLEDLIRCAAIPSLLNAACSEQDSEPGARLTCRILLHLFKTPQLNPCQSDGNKPTVGIRSSCDRHLLAASQNRIVDGAVFAVLKAVFVLGDAELKGSGFTVTGGTEELPEEEGGGGSGGRRQGGRNISVETASLDVYAKYVLRSICQQEWVGERCLKSLCEDSNDLQDPVLSSAQAQRLMQLICYPHRLLDNEDGENPQRQRIKRILQNLDQWTMRQSSLELQLMIKQTPNNEMNSLLENIAKATIEVFQQSAETGSSSGSTASNMPSSSKTKPVLSSLERSGVWLVAPLIAKLPTSVQGHVLKAAGEELEKGQHLGSSSRKERDRQKQKSMSLLSQQPFLSLVLTCLKGQDEQREGLLTSLYSQVHQIVNNWRDDQYLDDCKPKQLMHEALKLRLNLVGGMFDTVQRSTQQTTEWAMLLLEIIISGTVDMQSNNELFTTVLDMLSVLINGTLAADMSSISQGSMEENKRAYMNLAKKLQKELGERQSDSLEKVRQLLPLPKQTRDVITCEPQGSLIDTKGNKIAGFDSIFKKEGLQVSTKQKISPWDLFEGLKPSAPLSWGWFGTVRVDRRVARGEEQQRLLLYHTHLRPRPRAYYLEPLPLPPEDEEPPAPTLLEPEKKAPEPPKTDKPGAAPPSTEERKKKSTKGKKRSQPATKTEDYGMGPGRSGPYGVTVPPDLLHHPNPGSITHLNYRQGSIGLYTQNQPLPAGGPRVDPYRPVRLPMQKLPTRPTYPGVLPTTMTGVMGLEPSSYKTSVYRQQQPAVPQGQRLRQQLQQSQGMLGQSSVHQMTPSSSYGLQTSQGYTPYVSHVGLQQHTGPAGTMVPPSYSSQPYQSTHPSTNPTLVDPTRHLQQRPSGYVHQQAPTYGHGLTSTQRFSHQTLQQTPMISTMTPMSAQGVQAGVRSTAILPEQQQQQQQQQQQQQQQQQQQQQQQQQQYHIRQQQQQQILRQQQQQQQQQQQQQQQQQQQQQQQQQQHQQQQQQQAAPPQPQPQSQPQFQRQGLQQTQQQQQTAALVRQLQQQLSNTQPQPSTNIFGRY

(19) United States
(12) Patent Application Publication
(10) Pub. No.: US 2026/0248803 A1
(43) Pub. Date: Aug. 27, 2026

(54) METHODS OF TREATING UTERINE FIBROIDS

(71) Applicant: Northwestern University, Evanston, IL (US)

(72) Inventors: Ping Yin, Evanston, IL (US); Serdar E. Bulun, Evanston, IL (US)

(21) Appl. No.: 19/550,179

(22) Filed: Feb. 25, 2026

Related U.S. Application Data
(60) Provisional application No. 63/763,250, filed on Feb. 25, 2025.

(51) Int. Cl. A61K 31/541; A61K 31/18; A61K 31/255; A61K 31/44; A61K 31/4985; A61K 31/501; A61K 31/506; A61K 31/519; A61P 35/00

(52) U.S. Cl. CPC A61K 31/541; A61K 31/18; A61K 31/255; A61K 31/44; A61K 31/4985; A61K 31/501; A61K 31/506; A61K 31/519; A61P 35/00

(57) ABSTRACT

Disclosed herein are methods of treating uterine leiomyomas in a subject in need thereof comprising administering a STAT3 pathway inhibitor to the subject, methods of killing a tumor cell comprising contacting the tumor cell with a STAT3 pathway inhibitor, and methods of reducing the likelihood of recurrence of uterine leiomyomas in a subject in need thereof comprising administering a STAT3 pathway inhibitor.

### CROSS-REFERENCE TO RELATED APPLICATIONS

The present application claims priority to U.S. Provisional Patent Application No. 63/763,250 that was filed Feb. 25, 2025, the entire contents of which are hereby incorporated by reference.

### STATEMENT REGARDING FEDERALLY SPONSORED RESEARCH

This invention was made with government support under grant number CA298456 awarded by the National Institutes of Health. The government has certain rights in the invention.

### REFERENCE TO A SEQUENCE LISTING

A Sequence Listing accompanies this application and is submitted as an xml file of the sequence listing named “702581_02759.xml” which is 3,873 bytes in size and was created on Feb. 18, 2026. The sequence listing is electronically submitted via Patent Center and is incorporated by reference herein in its entirety.

### FIELD OF THE INVENTION

The disclosed technology is generally directed to the field of medical oncology. More particularly the technology is directed to treatment of uterine leiomyomas, also referred to as uterine fibroids.

### BACKGROUND OF THE INVENTION

Uterine leiomyomas (LMs, fibroids) affect 70-80% of women, with 15-30% experiencing significant symptoms. There is currently no long-term or effective medical treatment available, and many women opt to undergo hysterectomy. Understanding how LMs develop, grow, and become fibrotic is essential for identifying new non-surgical treatments for the disease. Accordingly, new technology for treating uterine fibroids is needed in the art.

### BRIEF SUMMARY OF THE INVENTION

In an aspect of the current disclosure, methods of treating uterine leiomyomas (LMs) in a subject in need thereof are provided. In some embodiments, the method comprising administering a therapeutically effective amount of a signal transducer and activator of transcription 3 (STAT3) pathway inhibitor to the subject to treat the LMs in the subject. In some embodiments, the STAT3 pathway inhibitor comprises a STAT3 specific inhibitor or a Janus kinase (JAK) inhibitor. In some embodiments, the STAT3 specific inhibitor is S3I-201, C188-9, WP1066, or VVD-130850. In some embodiments, the JAK inhibitor is selected from abrocitinib, fedratinib, deucravacitinib, delgocitinib, baricitinib, upadacitinib, ruxolitinib, tofacitinib, and filgotinib. In some embodiments, the JAK inhibitor is selected from abrocitinib, fedratinib, deucravacitinib, and delgocitinib. In some embodiments, the LMs in the subject comprise at least one mutation in mediator complex subunit 12 (MED12). In some embodiments, the mutation in MED12 comprises a mutation that alters MED12 function and inactivates CDK8/19 kinase activity. In some embodiments, the mutation in MED12 comprises a mutation in G44, as compared to SEQ ID NO: 1. In some embodiments, the mutation in G44 is a G44D substitution mutation. In some embodiments, the method upregulates an apoptosis gene signature in the cells of the LMs or inhibits proliferation of the cells of the LMs. In some embodiments, treating LMs in a subject in need thereof comprises reducing the likelihood of recurrence of LMs in the subject.

In an aspect of the current disclosure, methods of killing or inhibiting proliferation of a tumor cell comprising a mutation in mediator complex subunit 12 (MED12) are provided. In some embodiments, the methods comprise contacting the tumor cell with an effective amount of a signal transducer and activator of transcription 3 (STAT3) pathway inhibitor to kill or inhibit proliferation of the tumor cell. In some embodiments, the STAT3 pathway inhibitor comprises a STAT3 specific inhibitor or a Janus kinase (JAK) inhibitor. In some embodiments, the STAT3 specific inhibitor is S3I-201, C188-9, WP1066, or VVD-130850. In some embodiments, the JAK inhibitor is selected from abrocitinib, fedratinib, deucravacitinib, delgocitinib, baricitinib, upadacitinib, ruxolitinib, tofacitinib, and filgotinib. In some embodiments, the JAK inhibitor is selected from abrocitinib, fedratinib, deucravacitinib, and delgocitinib. In some embodiments, the tumor cell is a uterine leiomyoma (LM) cell. In some embodiments, the mutation in MED12 comprises a mutation that alters MED12 function and inactivates CDK8/19 kinase activity. In some embodiments, the mutation in MED12 comprises a G44 mutation, with reference to SEQ ID NO: 1. In some embodiments, the G44 mutation is a G44D substitution mutation. In some embodiments, the method upregulates an apoptosis gene signature in the cells of the LMs.

In an aspect of the current disclosure, methods of reducing the likelihood recurrence of uterine leiomyomas (LMs) in a subject in need thereof are provided. In some embodiments, the methods comprise administering a therapeutically effective amount of a signal transducer and activator of transcription 3 (STAT3) pathway inhibitor to the subject to reduce the likelihood of recurrence of the uterine leiomyomas in the subject. In some embodiments, the subject has been treated with a surgical therapy, a radiation therapy, or a hormone therapy. In some embodiments, the hormone therapy comprises a gonadotropin-releasing hormone (GnRH) analogue. In some embodiments, the GnRH analogue comprises goserelin, nafarelin, buserelin, or leuprorelin. In some embodiments, the STAT3 pathway inhibitor comprises a STAT3 specific inhibitor or a Janus kinase (JAK) inhibitor. In some embodiments, the STAT3 specific inhibitor is S3I-201, C188-9, WP1066, or VVD-130850. In some embodiments, the JAK inhibitor is selected from abrocitinib, fedratinib, deucravacitinib, delgocitinib, baricitinib, upadacitinib, ruxolitinib, tofacitinib, and filgotinib. In some embodiments, the JAK inhibitor is selected from abrocitinib, fedratinib, deucravacitinib, and delgocitinib. In some embodiments, the LMs in the subject comprise at least one mutation in mediator complex subunit 12 (MED12). In some embodiments, the mutation in MED12 comprises a mutation that alters MED12 function and inactivates CDK8/19 kinase activity. In some embodiments, the mutation in MED12 comprises a G44 mutation, with reference to SEQ ID NO: 1. In some embodiments, the G44 mutation is a G44D substitution mutation. In some embodiments, the method upregulates an apoptosis gene signature in the cells of the LMs.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Non-limiting embodiments of the present invention will be described by way of example with reference to the accompanying figures, which are schematic and are not intended to be drawn to scale. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention.

FIG. 1 shows an overall hypothesis and aims. S: serine 727; Y: tyrosine 705; P: phosphorylation.

FIGS. 2A, 2B, 2C, and 2D show Mut-MED12 cells are more sensitive to STAT3 inhibition than wt-MED12 and MyoF cells. (A,B) Primary cells from patient-matched MyoF, mut-MED12 -LM, and wt-MED12 LM were treated with vehicle (Veh) or STAT3-specific inhibitors S3I-201 (STAT3i-1, A) or C188-9 (STAT3i-2, B) at the indicated doses for 72 h and analyzed with CCK-8 for cell viability (n=3 patients). (C,D) The same treatments were used to compare the effects of STAT3 inhibition on mut- and wt-MED12 UtSM cell line viability (n=3). *P<0.05, **P<0.01, ***P<0.005, #P<0.0001.

FIGS. 3A, 3B, 3C, and 3D show MyoF adjacent to mut-MED12 LM express STAT3-activating cytokines that promote mut-MED12 LM cell growth. (A) GSEA analysis of tissue RNA-seq data revealed enrichment of inflammatory response pathway in MyoF adjacent to mut-vs. wt-MED12 LM (n=10). (B) MyoF adjacent to mut-MED12 LM exhibited the highest expression levels of the indicated cytokines (n=20). (C) OSM, LIF, and IL1β (100, 50, and 10 ng/ml, respectively) treatment for 48 or 72 h increased mut-MED12 LM cell viability (n=5). (D) OSM phosphorylated JAK1, JAK2, TYK2, and Y⁷⁰⁵-STAT3 in mut-MED12 LM cells. *P<0.05, **P<0.01, ^{#}P<0.0001.

FIGS. 4A, 4B, 4C, and 4D show STAT3 regulates the expression of genes key for mut-MED12 LM growth. (A) Heatmap shows differentially expressed genes (fold change >2, P-adj<0.05) between Veh- and STAT3i (S3I-201)-treated primary mut-MED12 LM cells (n=9). (B) GO (Panther) pathways enriched in up- and downregulated genes (FDR<0.05). (C) GSEA analysis identified the apoptosis pathway to be enriched in STAT3i-upregulated genes. (D) Heatmaps show pro-apoptotic genes downregulated in mut-MED12 LM vs. MyoF tissues (n=12) but upregulated by STAT3i in mut-MED12 LM primary cells. *P<0.05, **P<0.01, #P<0.0001.

FIGS. 5A and 5B show STAT3 inhibition suppresses collagen expression in mut-MED12 LM cells. (A) Heatmaps show significantly upregulated collagen genes in mut-MED12 LM vs. MyoF tissues (n=12), that were also inhibited by STAT3i in primary mut-MED12 LM cells (n=9). (B) STAT3 siRNA inhibits COL1A1 and COL3A1 expression in mut-MED12 LM cells. *P<0.05, **P<0.01, ***P<0.005, ^{#}P<0.0001.

FIGS. 6A, 6B, and 6C show CDK8/19 activity-dependent STAT3-target genes are involved in mut-MED12 LM growth. (A) Integrative analyses of RNA-seq data (top) from primary wt-MED12 LM cells treated with CDK8/19 inhibitor (CDK8i) SEL120+/−STAT3i S3I-201 (n=6) and RNA-seq data (bottom) from wt- and mut-MED12 LM and MyoF tissues (n=20). Venn-diagram and heatmap show 245 genes upregulated by CDK8i but reversed by STAT3i in wt-MED12 LM cells and also overexpressed specifically in mut-MED12 LM tissue (adjusted P<0.05). (B,C) Bar graphs show the top 5 of 245 genes upregulated by CDK8i but reversed by STAT3i in wt-MED12 LM cells (B) that are also expressed at the highest levels in mut-MED12 tissues (C). *P<0.05, **P<0.01, ***P<0.005, ^{#}P<0.0001.

FIG. 7 shows a heatmap of the Pearson correlation between the mRNA levels of selected CDK8/19 activity-dependent STAT3-target genes (ADAM12, KRT17, TDO2, STA8SIA2, and MMP11) and those of candidate genes (COL1A1, COL1A2, COL3A1, CCND1, IGF2, and TGFß3) key for LM growth in mut-MED12 LM, wt-MED12 LM, and MyoF (n=20). The number in each cell indicates Pearson's R.

FIG. 8 shows representative images of spatial transcriptomics data. Images show specific overexpression of KRT17, TDO2, and ADAM12 in mut-vs. wt-MED12 LM and MyoF (n=16).

FIGS. 9A and 9B show STAT3 specifically regulates ADAM12 expression in primary mut-MED12 LM cells. (A) Two STAT3 inhibitors (STAT3i-1, -2), but not inhibitors of STAT1 (li, Fludarabine), 5 (5i, CAS285986-31-4), or 6 (6i, AS1517499) inhibit ADAM12 expression (n=3 patients). (B) STAT3 siRNA, but not siRNA for STAT1, 5a, 5b, or 6, inhibits ADAM12 expression (n=3). *P<0.05, **P<0.01, ^{#}P<0.0001.

FIGS. 10A, 10B, 10C, and 10D show STAT3 DNA binding and transcriptional activities are higher in mut-vs wt-MED12 cells. (A) STAT3-responsive luciferase activity was significantly higher in mut-vs wt-MED12 UtSM cells. (B) Genome browser view of STAT3 and H3K27ac binding sites in ADAM12 gene locus in MCF10A cells (ENCODE data). P1 to P3 indicates the location of ChIP-qPCR primers used in (C,D). (C,D) ChIP-qPCR assays show higher binding of STAT3 (C) and H3K27ac (D) in ADAM12 gene locus in mut-vs wt-MED12 LM. TSS: transcription start site. *P<0.05, **P<0.01, ^{#}P<0.0001.

FIGS. 11A, 11B, and 11C show MED12-dependent CDK8/19 activity alters the STAT3 S⁷²⁷ and Y⁷⁰⁵ phosphorylation ratio. (A) In vitro kinase assays reveal direct S⁷²⁷ phosphorylation of STAT3 by Mediator kinase module containing wt- but not mut (G44D)-MED12. Left: Coomassie blue staining of SDS-PAGE-purified Mediator kinase module containing CCNC, CDK8, MED13, and wt- or mut-MED12. Right: Kinase assays were performed by incubating Mediator kinase module bearing wt- or mut-MED12 with wt- or phospho-mutant-(S727A) STAT3 or STAT1 substrates (aa710-750 from the STAT1/3 C-terminus). Kinase reactions were resolved by SDS-PAGE and subjected to Phosphoimager analysis to detect phosphorylated (³²P) substrates (top bands) or Coomassie blue staining to visualize input substrates (bottom bands). (B,C) Immunoblot image (B) and quantification (C) show that pretreatment with CDK8i for 30 min reciprocally decreased pS⁷²⁷-STAT3 but increased pY⁷⁰⁵-STAT3 levels induced by IL6 in primary wt-MED12 LM cells (n=3 patients). min: minutes. **P<0.01.

FIG. 12 shows representative immunofluorescent confocal microscopic images show lower pS⁷²⁷-STAT3 level (top) but higher pY⁷⁰⁵-STAT3 (top) and TDO2 (middle) levels in mut-vs wt-MED12 primary LM cells (n=3). Merged staining of TDO2, p-STAT3, and DAPI (blue, not separately shown) were depicted at the bottom. Large yellow inserts show enlarged cells in small inserts.

FIGS. 13A and 13B show mut-MED12 decreases pS⁷²⁷-STAT3 and pS⁷²⁷-STAT1 levels but increases only pY⁷⁰⁵-STAT3 levels as detected by IB of whole cell lysates isolated from (A) and immunostaining of (B) mut- and wt-MED12 UtSM cells.

FIGS. 14A, 14B, and 14C show PGR stimulates STAT3 expression. (A) UCSC genome browser shows PGR binding to the STAT3 promoter in LM (n=5). (B,C) PGR siRNA decreases STAT3 mRNA (B) and protein (C) levels. Data from mut- and wt-MED12 LM cells were analyzed together because of the similar response to PGR siRNA (n=6). TSS: transcription start site. *P<0.05, ***P<0.005.

FIG. 15 shows experimental design of Aim 2A. C188-9, STAT3 inhibitor.

### DETAILED DESCRIPTION OF THE INVENTION

Disclosed herein are methods of treating uterine leiomyomas (LMs) in a subject in need thereof, methods of killing a tumor cell, and methods of reducing the likelihood recurrence of uterine leiomyomas (LMs) in a subject in need thereof.

### Methods of Treating Uterine Leiomyomas in a Subject

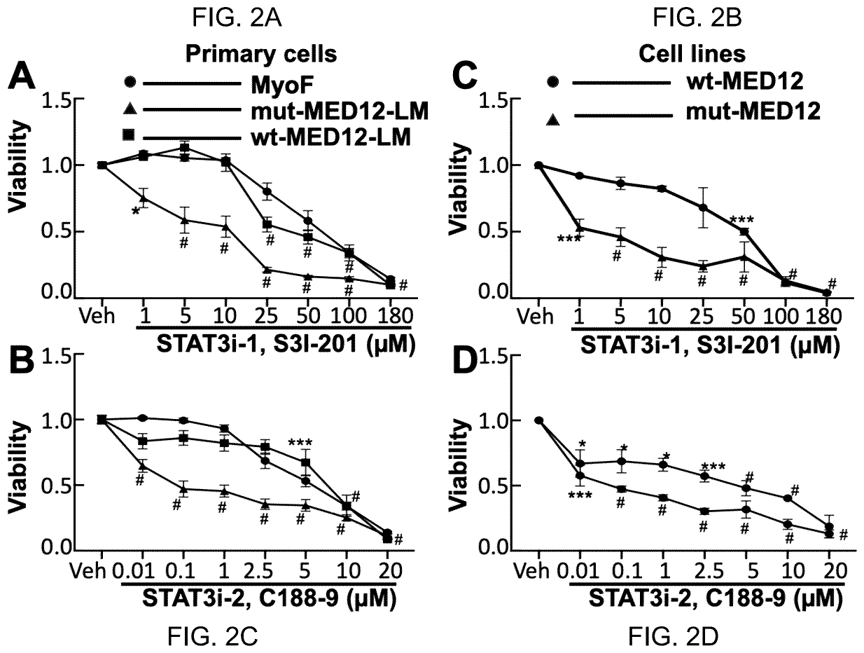

The inventors discovered that STAT3 inhibition blocks steroid hormone-dependent LM growth (FIG. 2). Therefore, in some embodiments, methods of treating uterine leiomyomas in a subject in need thereof are provided and comprise administering a therapeutically effective amount of a signal transducer and activator of transcription 3 (STAT3) pathway inhibitor to the subject to treat the LMs in the subject. As used herein, a “STAT3 pathway inhibitor” refers to a compound that inhibits the function of STAT3 by affecting STAT3 function directly or by affecting upstream or downstream signaling of STAT3.

STAT3 may be targeted specifically for inhibition using, e.g., a STAT3 specific inhibitor. Exemplary STAT3 specific inhibitors include, but are not limited to, S3I-201, C188-9, WP1066, or VVD-130850.

As used herein, “S3I-201” refers to a compound having the following chemical structure:

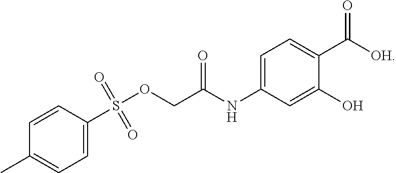 [SMILES: Cc1ccc(S(=O)(=O)OCC(=O)Nc2ccc(C(=O)O)c(O)c2)cc1]

As used herein, “C188-9” refers to a compound having the following chemical structure:

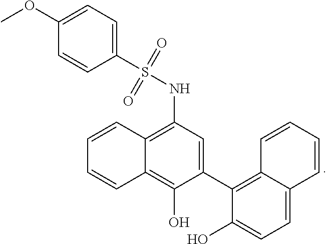 [SMILES: COc1ccc(S(=O)(=O)Nc2cc(-c3c(O)ccc4ccccc34)c(O)c3ccccc23)cc1]

As used herein, “WP1066” refers to a compound having the following chemical structure:

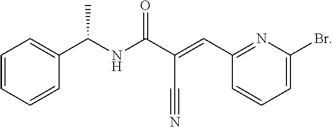 [SMILES: C[C@H](NC(=O)/C(C#N)=C/c1cccc(Br)n1)c1ccccc1]

In other embodiments, the STAT3 pathway inhibitor may comprise a Janus kinase (JAK) inhibitor. Exemplary JAK inhibitors may include, but are not limited to, abrocitinib, fedratinib, deucravacitinib, delgocitinib, baricitinib, upadacitinib, ruxolitinib, tofacitinib, or filgotinib.

The protein mediator complex subunit 12 (MED12) is commonly mutated in uterine leiomyomas. The inventors discovered that STAT3 activation by CDK8/19 disruption is a key mediator of mut-MED12-driven LM growth. Therefore, in some embodiments, the LMs in the subject comprises a mutation in MED12, e.g., a mutation that alters MED12 function and inactivates CDK8/19 kinase activity. Methods for detecting mutations that alter MED12 function and inactivates CDK8/19 kinase activity are known in the art and include, but are not limited to, sequencing and functional testing of mutant MED12.

SEQ ID NO: 1 is the wild-type sequence of human MED12. A mutation at position 44 (G44) of MED12 is commonly found in LMs. Therefore, the LMs may comprise a mutation in G44 of MED12. The mutation may be, e.g., a substitution mutation, e.g., a G44D substitution mutation.

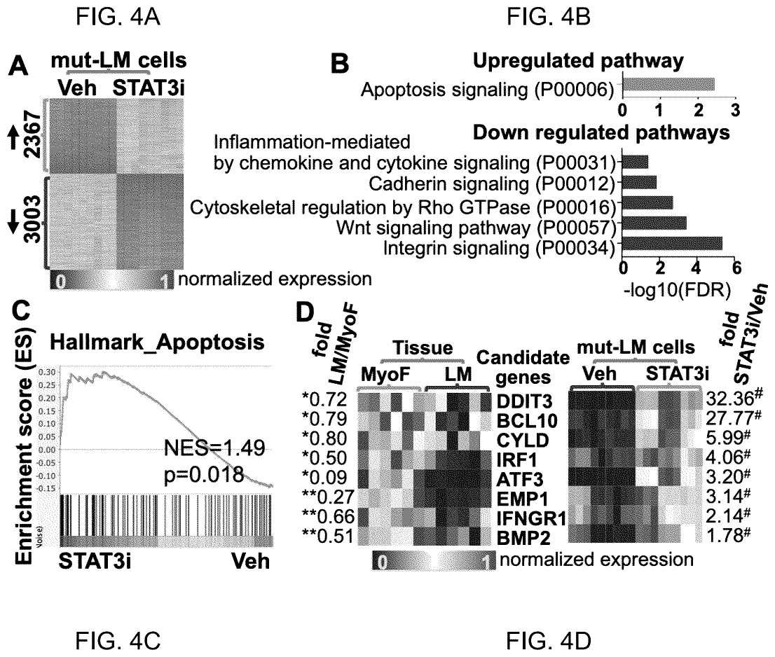

Referring now to FIGS. 4A, 4B, and 4C, the method may upregulate an apoptosis gene signature in the cells of the LMs, as demonstrated by the inventors. Apoptosis signatures may be measured by methods known in the art, e.g., next generation sequencing coupled with pathway analysis, gene set enrichment analysis (GSEA) or protein analysis through evolutionary relationships (PANTHER) analysis, e.g., as shown in FIG. 4.

In some embodiments, the methods may reduce the likelihood of recurrence of LMs in the subject. Methods of measuring recurrence of LMs in a subject are known in the art and include radiological assessments and physical assessments following successful treatment of the subject, which may comprise remission or a complete response to another therapy, e.g., a surgical therapy, a radiation therapy, an immunotherapy, or a hormone therapy. The hormone therapy may comprise a gonadotropin-releasing hormone (GnRH) analogue, e.g., goserelin, nafarelin, buserelin, or leuprorelin.

Combination therapies are also provided, wherein the disclosed methods further comprise administering an additional therapy, e.g., a surgical therapy, a radiation therapy, an immunotherapy, or a hormone therapy.

As used herein, a “therapeutically effective amount” or an “effective amount” refers to the amount of an agent to cause a desired effect in a subject. In one example, a therapeutically effective amount is an amount that is effective in reducing at least one sign or symptom of uterine leiomyoma in a subject, e.g., at least one of a reduction in tumor volume, a reduction in tumor number, a reduction in tumor markers in the blood, a reduction in tumor cell-free DNA (cfDNA), a reduction in pain, a reduction in perceived abdominal pressure, and a reduction in abnormal vaginal bleeding.

Similarly, “treating,” uterine leiomyomas refers to reducing or improving at least one sign or symptom of uterine leiomyomas in a subject, e.g., at least one of a reduction in tumor volume, a reduction in tumor number, a reduction in tumor markers in the blood, a reduction in tumor cell-free DNA (cfDNA), a reduction in pain, a reduction in perceived abdominal pressure, and a reduction in abnormal vaginal bleeding.

As used herein, “administering” may refer to administration by any suitable route as determined by a physician based on the identity of the compound being administered and other relevant factors, e.g., oral, sublingual, intravenous, intramuscular, intrathecal, or subcutaneous administration. Administration may be performed according to any suitable schedule at the discretion of a physician, e.g., once daily, twice daily, three times daily, four times daily. Administration may be performed for a time period of about one day to about 1 year, or more. Administration may be performed for, e.g., about one day, two days, three days, four days, five days, six days, seven days, eight days, nine days, ten days, eleven days, twelve days, thirteen days, fourteen days, or more. Administration may be performed for about one week, two weeks, three weeks, four weeks, one month, two months, three months, four months, five months, six months, seven months, eight months, nine months, ten months, eleven months, twelve months, or more, or any subrange or value therein.

As used herein, a “subject in need thereof” refers to a subject in need of treatment by the disclosed methods, e.g., a subject suffering from uterine leiomyoma (which may refer to a subject suffering from a single “fibroid” or multiple “fibroids”) or a subject that was treated for uterine leiomyomas and is in need of prevention of recurrence of the disease.

### Methods of Killing or Inhibiting Proliferation a Tumor Cell

In an aspect of the current disclosure, methods of killing or inhibiting proliferation of a tumor cell are provided. The methods comprising contacting the tumor cell with an effective amount of a signal transducer and activator of transcription 3 (STAT3) pathway inhibitor to kill or inhibit proliferation of the tumor cell. The tumor cell may comprise a uterine leiomyoma cell. The cell may comprise a mutation in mediator complex subunit 12 (MED12), e.g., a G44 mutation, with reference to SEQ ID NO: 1. The G44 mutation may be a G44D mutation.

Methods of monitoring and detecting the killing of cells, e.g., tumor cells, are known in the art. For example, the killing of tumor cells in vitro may be measured by the use of live cell dyes, e.g., propidium iodide or others. The killing of cells in vivo may be measured by imaging, e.g., computer-aided tomography, magnetic resonance imaging, positron emission tomography, etc. As used herein, a “effective amount,” in the context of methods of killing a tumor cell, refers to an amount that induces death of the tumor cell. Death of the tumor cell may include, but is not limited to, apoptosis. Reagents for measuring apoptosis are known in the art and include, e.g., PARP cleavage detection reagents and caspase detection reagents. Alternatively, induction of cell death, e.g., through apoptosis, may be measured in the transcriptome of cells as described above.

### Methods of Reducing the Likelihood of Recurrence of Uterine Leiomyomas

In an aspect of the current disclosure, methods of reducing the likelihood recurrence of uterine leiomyomas (LMs) in a subject in need thereof are provided, the methods comprising administering a therapeutically effective amount of a signal transducer and activator of transcription 3 (STAT3) pathway inhibitor to the subject to reduce the recurrence of uterine leiomyomas in the subject.

### Miscellaneous

Unless otherwise specified or indicated by context, the terms “a”, “an”, and “the” mean “one or more.” For example, “a molecule” should be interpreted to mean “one or more molecules.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean plus or minus ≤10% of the particular term and “substantially” and “significantly” will mean plus or minus >10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion additional components other than the components recited in the claims. The term “consisting essentially of” should be interpreted to be partially closed and allowing the inclusion only of additional components that do not fundamentally alter the nature of the claimed subject matter.

All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

All references, including publications, patent applications, and patents, cited herein are hereby incorporated by reference to the same extent as if each reference were individually and specifically indicated to be incorporated by reference and were set forth in its entirety herein.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### EXAMPLES

### Example 1—Targeting STAT3 in Uterine Fibroids

Uterine leiomyomas (LMs, fibroids) are the most important neoplastic threat to women worldwide. As no long-term non-invasive treatment option exists for LMs, deeper insight into tumor etiology is key to develop more effective medical therapies. Accordingly, this proposal is impactful as it suggests a novel etiological basis for the predominant MED12-mutated LM subtype and offers proof of concept for therapeutic intervention in this specific genetic setting. LMs arise from recurrent and mutually exclusive genetic alterations in a limited number of driver genes. Among these, mutations in the RNA Polymerase II Mediator subunit MED12 (mut-MED12) are by far the most prevalent, accounting for 77.4% of LMs. Recently, the inventors showed that LM driver mutations in MED12 disrupt CDK8/19 kinase activity in Mediator, revealing the first and heretofore only known biochemical defect arising from these pathogenic mutations and further implying a new etiological role for CDK8/19 in LM pathogenesis. Herein, the inventors identify Signal transducer and activator of transcription 3 (STAT3), a transcription factor implicated in tumor growth and fibrosis, as a direct MED12-dependent CDK8/19 substrate. Physiologically, the inventors show that CDK8/19, by direct phosphorylation of STAT3 serine 727, is a negative regulator of STAT3 transcriptional activity. However, the role of the STAT3 pathway in LM growth and whether this activity is altered by mut-MED12 remains unexplored. The inventors' preliminary data indicate that STAT3 activation by CDK8/19 disruption is a key mediator of mut-MED12-driven LM growth. (i) Compared with wild-type (wt)-MED12 cells, mut-MED12 primary LM cells or a human myometrial smooth muscle cell line with CRISPR-engineered mut-MED12 responded to STAT3 inhibitors with greater sensitivity and decreased growth. (ii) CDK8/19 disruption by mut-MED12 reduced STAT3 serine 727 phosphorylation, leading to an increase in tyrosine 705 phosphorylation and strikingly enhancing the transcriptional activity and chromatin binding properties of STAT3 that drives expression of genes crucial for LM growth. (iii) STAT3-activating cytokines were specifically upregulated in myometrium adjacent to mut-MED12 LM, activated the JAK/STAT3 pathway, and promoted mut-MED12 LM cell growth. (iv) Progesterone receptor binds to the STAT3 gene promoter and stimulates its expression. The inventors hypothesize that Mediator kinase disruption as a consequence of LM driver mutations in MED12 alters the phosphorylation pattern of STAT3 to favor a hyperactive form, one with enhanced chromatin binding properties leading to transcriptional activation of unique set of genes responsible for LM growth. The inventors propose that STAT3 inhibition, e.g., by using FDA-approved or in-pipeline agents targeting the JAK/STAT3 pathway, will block steroid hormone-dependent LM growth, especially for the mut-MED12 subtype. Using a clinically relevant patient-derived xenograft LM mouse model and a cutting-edge single-nucleus multiomics approach, the inventors will: (1) determine the functional role of the STAT3 pathway in LM growth in vivo; and (2) define the cell populations with unique epigenetic and transcriptomic processes responsible for STAT3-driven growth in mut-MED12 LM and verify the mechanistic findings in clinical samples.

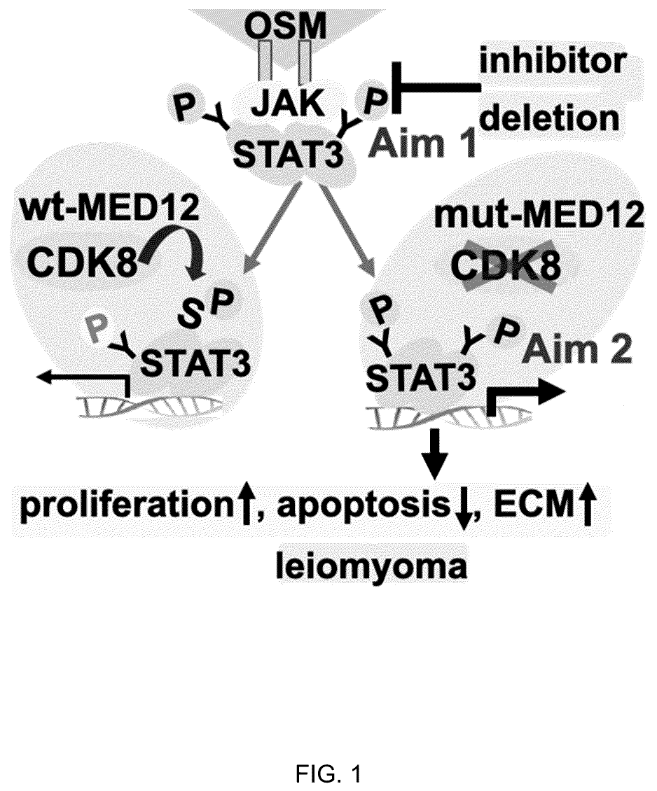

Uterine leiomyomas (LMs, fibroids) affect 70-80% of women, with 15-30% experiencing significant symptoms. There is currently no long-term or effective medical treatment available, and many women opt to undergo hysterectomy. Understanding how LMs develop, grow, and become fibrotic is essential for identifying new non-surgical treatments for the disease. Accordingly, this proposal is impactful as it suggests a novel etiological basis for LM and further offers proof of concept for therapeutic intervention in this specific genetic setting. LMs arise from recurrent and mutually exclusive genetic alterations in a limited number of driver genes. Among these, mutations in the RNA Polymerase II Mediator subunit MED12 (mut-MED12) are by far the most prevalent, accounting for 77.4% of LMs. Recently, the inventors showed that LM driver mutations in MED12 disrupt CycC-CDK8/19 kinase activity in Mediator, revealing the first and heretofore only known biochemical defect arising from these pathogenic mutations and further implying a new etiological role for CDK8/19 in LM pathogenesis. Herein, the inventors identify Signal transducer and activator of transcription 3 (STAT3), a transcription factor implicated in tumor growth and fibrosis, as a direct MED12-dependent CDK8/19 substrate. Physiologically, the inventors show that CDK8/19, by direct phosphorylation of STAT3 serine 727, is a negative regulator of STAT3 transcriptional activity. However, the role of the STAT3 pathway in LM growth and whether this activity is altered by mut-MED12 remains unexplored. The inventors' data suggest that STAT3 hyperactivation by CDK8/19 disruption is a key mediator of mut-MED12-driven LM growth. (i) Compared with wild-type (wt)-MED12 cells, mut-MED12 primary LM cells or a human myometrial smooth muscle cell line with CRISPR-engineered mut-MED12 responded to STAT3 inhibitors with greater sensitivity and decreased growth. (ii) Mediator kinase disruption by mut-MED12 reduced STAT3 serine 727 phosphorylation, leading to an increase in tyrosine 705 phosphorylation, strikingly enhancing its transcriptional activity and chromatin binding properties that drive expression of genes crucial for LM growth. (iii) STAT3-activating cytokines, such as oncostatin M (OSM), were specifically upregulated in myometrium (MyoF) adjacent to mut-MED12 LM, activated the JAK-STAT3 pathway, and promoted mut-MED12 LM cell growth. (iv) Progesterone receptor binds to the STAT3 gene promoter and stimulates its expression. Thus, the activated STAT3 pathway appears to be crucial for ovarian steroid hormone-dependent LM growth and may be a therapeutic target, especially for mut-MED12 LM. The goals of this project are to delineate the role of STAT3 hyperactivation in response to Mediator kinase disruption in mut-MED12 LM growth, and to assess whether STAT3 is a clinically beneficial therapeutic target. Overall, the inventors hypothesize that Mediator kinase disruption as a consequence of LM driver mutations in MED12 alters the phosphorylation pattern of STAT3 to favor a hyperactive form, one with enhanced chromatin binding properties leading to transcriptional activation of genes responsible for LM growth. Accordingly, the inventors propose that STAT3 inhibition using FDA-approved or in-pipeline agents targeting the JAK/STAT3 pathway will block steroid hormone-dependent LM growth, especially for the mut-MED12 subtype (FIG. 1). To test these hypotheses, the inventors propose the following aims, which address multiple NIH priority research areas.

### AIM 1. Determine the Functional Role of the STAT3 Pathway in LM Growth In Vivo.

Using a clinically relevant patient-derived xenograft LM mouse model, the inventors will determine whether pharmacologically or genetically blocking the JAK/STAT3 pathway activity inhibits LM cell proliferation and ECM production, induces apoptosis, and reduces tumor growth preferentially in the mut-MED12 LM subtype. The inventors will investigate whether STAT3 activation by OSM preferentially enhances mut-MED12 LM growth. Additionally, the inventors will determine whether depletion of endogenous CDK8/19-mediated STAT3 target genes preferentially inhibits mut-MED12 LM growth.

AIM 2. Define the Cell Populations with Unique Epigenetic and Transcriptomic Processes Responsible for STAT3-Driven Growth in Mut-MED12 LM and Verify the Mechanistic Findings in Clinical Samples.

Using a cutting-edge multiomics approach, the inventors will perform parallel single-nucleus (sn) RNA-seq, snATAC-seq and MED12-targeted long-read sequencing on mut-MED12 LM, wt-MED12 LM, and matched MyoF patient tissues, and their corresponding xenografted tissues treated with ovarian steroid hormones with or without STAT3 inhibitor. The inventors will identify gene expression signatures dysregulated specifically in mut-MED12 LM cells and elucidate their epigenetic mechanisms at the single-cell level. Furthermore, the inventors will determine the in vivo relevance of the hyperactive STAT3 pathway in mut-MED12 LM pathology by assessing the correlation between the levels of cell proliferation and fibrotic gene markers and STAT3 phosphorylation status and its target gene expression in whole tissues of mut- and wt-MED12 LM and matched MyoF obtained from patient specimens.

This will determine the role of hyperactive STAT3 as a key mediator of CDK8/19 activity loss that promotes mut-MED12 LM growth. The inventors' work will pave the way for translational research and the development of precision medicine therapeutics targeting the mut-MED 12 subtype that accounts for 77.4% of all LMs.

### Research Strategy

### A. Significance

### LM Pathogenesis and Treatment Barriers.

Uterine leiomyomas (LMs or fibroids) are the most common tumor in women and represent one of the most important public health problems worldwide¹. By the age of 50 years, up to 80% of all women will have developed at least one LM, and 15% to 30% will develop severe symptoms^{2, 3}. These smooth muscle tumors disrupt uterine function and cause excessive uterine bleeding, anemia, obstruction of labor, and urinary incontinence⁴⁻⁷. African American women develop larger LM at a higher rate and at earlier ages, and develop more severe symptoms than European American women⁸⁻¹¹. Approximately 250,000 hysterectomies or myomectomies, and many less invasive but morbid procedures, are performed in the US annually to remove LM, costing up to $34.4 billion¹². Although LM growth depends on estrogen (E2) and progesterone (P4) acting through their receptors (ESR1 and PGR, respectively), the action of cytokines, growth factors, genetic mutations, and mechanical forces from the surrounding extracellular matrix (ECM) also play key roles¹³⁻¹⁸. This complex pathophysiology underlies the limited therapeutic efficacy of gonadotropin-releasing hormone (GnRH) analogues, the only class of pharmaceutics approved in the US for LM since the 1990s. GnRH analogues target ovarian hormone production but cause significant side effects and are not suitable for long-term use^{19, 20}. There is thus an urgent need for new medical options to treat LM.

### MED12 is a Dominant LM Driver.

LMs arise from recurrent and mutually exclusive genetic alterations in a limited number of driver genes^{17, 21-23}. Among these, mutations in the RNA Polymerase II Mediator subunit MED12 (mut-MED12) are by far the most prevalent, accounting for 77.4% of LMs^{17, 24}. Within Mediator, MED12 along with MED13, Cyclin C, and CDK8 (or its close paralog CDK19) comprise a large 4-subunit CDK8 Kinase Module (CKM) that confers positive and negative effects on transcription mediated through a 26-subunit Mediator core²⁵⁻³⁰ Previously, the inventors showed that LM driver mutations in MED12 disrupt CycC-CDK8 kinase activity through CDK8 T-loop destabilization, revealing the first and only known biochemical defect arising from these pathogenic mutations and identifying a new etiological role for Mediator kinase disruption in LM pathogenesis³¹⁻³⁵. The identity of key biological substrates downstream of CDK8/19 and how disruption of their phosphorylation as a consequence of LM driver mutations in MED12 affect LM growth, however, remain completely unknown.

### STAT3 is a Central Regulator of Fibrosis and Tumor Growth.

The inventors and others have identified members of the signal transduction and activator of transcription (STAT) pathway as substrates of CDK8/19 kinase activity³⁶⁻⁴⁰. Phosphorylation of STAT3 by growth factors and cytokines at various sites, specifically serine 727 (S⁷²⁷) and tyrosine 705 (Y⁷⁰⁵), modulates its transcriptional activity^{36, 41-49}. STAT3 phosphorylation at Y⁷⁰⁵ (pY⁷⁰⁵-STAT3) is known to mediate dimer formation crucial for cytokine-induced STAT3 transcriptional activity^{41, 45-47}. Conversely, CDK8 (or other kinase)-dependent phosphorylation of STAT3 at S⁷²⁷ (pS⁷²⁷-STAT3) has been reported to inhibit phosphorylation of neighboring Y⁷⁰⁵, shorten STAT3 chromatin interaction time, and suppress STAT3 transcriptional activity^{36, 41-44, 47}. The inventors' preliminary studies suggested that LM driver mutations in MED12 disrupt CDK8/19-dependent transcriptional inhibitory STAT3 phosphorylation at S⁷²⁷ leading to stimulatory phosphorylation at Y⁷⁰⁵ producing a STAT3 phosphor-species with enhanced chromatin binding that supports transcriptional activation of genes key for LM growth. STAT3 pathway plays pivotal roles in inflammation, fibrosis, and tumor growth by stimulating cell proliferation, promoting cell survival, activating fibroblasts, and enhancing ECM production^{47, 50-54} However, knowledge concerning the role of STAT3 in LM growth is limited.

### Scientific Premise and Impact:

Although genetic data unambiguously establish mut-MED12 as the predominant driver of 77.4% of LMs, the mechanistic basis for its tumorigenic activity is unknown yet key to the development of applied therapeutics^{17, 21, 23, 24, 55}. In this regard, the inventors previously established an essential role for wild type (wt)-MED12 in CDK8/19 activation that is disrupted by mut-MED12 in LM³¹⁻³⁵. In the inventors' preliminary work, the inventors identified STAT3 as a novel signaling hub at which MED12 mutation-induced CDK8/19 (kinase) disruption and steroid hormone action converge, suggesting STAT3 as a novel therapeutic target in LM, particularly the prevalent mut-MED12 subtype. First, the inventors found that mut-MED12 primary LM cells and CRISPR-engineered Gly44 mut-MED12-expressing human uterine smooth muscle (UtSM) cell lines were more sensitive than their corresponding wt-MED12 cells to STAT3 specific inhibitor-dependent growth restriction⁵⁶. Second, the inventors showed that mut-MED12-induced disruption of the kinase CDK8/19 leads to enhanced STAT3-dependent transcriptional activation of genes key for LM growth. Third, the inventors found that STAT3-activating cytokines, including oncostatin M (OSM) and leukemia inhibitor factor (LIF), were specifically upregulated in myometrium (MyoF) adjacent to mut-MED12 LM vs. MyoF adjacent to wt-MED12 LM, where they activated the janus kinase (JAK)-STAT3 pathway and promoted the growth of mut-MED12 LM cells. Fourth, the inventors showed that LM driver mutations in MED12 known to disrupt CDK8/19 (kinase) activity altered the ratio of functionally antagonistic Y⁷⁰⁵ and S⁷²⁷ phosphorylation to favor hyperactivated pY⁷⁰⁵-STAT3, revealing a mechanistic link between impaired Mediator kinase (CDK8/19) activity and enhanced chromatin binding and transcriptional properties of STAT3 in mut-MED12 LM. Finally, the inventors showed that PGR binds to the STAT3 gene promoter and stimulates its expression. Based on these findings, the inventors hypothesize that MED12 mutation-induced Mediator kinase disruption alters the phosphorylation pattern of STAT3 to favor a hyperactivated form-one with enhanced chromatin binding properties leading to transcriptional activation of a gene expression program that drives LM growth. Further, the inventors posit that STAT3 inhibition using FDA-approved or in-pipeline agents targeting the JAK/STAT3 pathway will block steroid hormone-dependent LM growth, especially the dominant mut-MED12 subtype (FIG. 1). These studies are uniquely significant because they address and mechanistically connect 2 overarching challenges facing the field: understanding the molecular pathogenesis of LMs and developing tolerable, fertility-compatible therapies for LMs. The inventors' studies also conform to multiple NICHD-defined priority areas: (i) transdisciplinary research and ‘omics approaches to gynecologic disorders; (ii) development of novel model systems to address gynecologic health and disease; and (iii) development of safe effective nonhormonal treatments for gynecologic conditions.

### B. Innovation

Conceptually, this disclosure is innovative on many fronts. First, the inventors' observation that mut-MED12-mediated Mediator kinase (CDK8/19) disruption enhances STAT3 pathway activity, which plays a critical role in tumor growth, has not been explored previously. This disclosure represents the first comprehensive analysis on this topic. Second, prior studies have unequivocally demonstrated that STAT3 is inappropriately hyperactivated in a high percentage of malignancies and fibrotic diseases, where it promotes the expression of genes stimulating cell proliferation, survival, inflammation, and ECM production^{47, 50-54}. The inventors' investigation into how Mediator kinase disruption because of MED12 LM driver mutations enhances STAT3-driven gene activation through altered phosphorylation represents the first mechanistic description of how hyperactive STAT3 drives mut-MED12 LM growth. Third, the inventors' studies identify STAT3 as a novel molecular target for the treatment of mut-MED12. Notably, extensive pre-clinical data have previously shown that STAT3 inhibition can reverse the abnormal behavior of fibrotic or tumor cells in vitro and in vivo; whereas normal cells can tolerate a loss of STAT3 function with few, if any, deleterious consequences^{52, 57-62}. Thus, STAT3 inhibition holds potential to have a high therapeutic index for LM treatment. This disclosure is innovative through its application of advanced single-nucleus multiomics analyses, including parallel single-nucleus (sn) RNA-seq/ATAC-seq and MED12-targeted sequencing, to an in vivo patient-derived LM xenograft model that can be manipulated including STAT3 inhibition and steroid hormone treatment. Combined with state-of-the-art computational data analysis pipelines, these tools will empower the inventors' efforts to clarify if and how STAT3 alters the epigenetic and transcriptomic state of single cell types to drive mut-MED12 LM growth.

### C. Approach

### 1. Preliminary Results

(1) STAT3 Inhibitors Decrease LM Cell Viability, with Greater Effects in Mut-MED12 vs. Wt-MED12 LM Cells.

Primary cells from mut-MED12 LM, wt-MED12 LM, and matched MyoF tissues were treated with various doses of 2 STAT3-specific inhibitors, S3I-201 and C188-9, and analyzed for cell viability (FIG. 2). Primary cells from mut-MED12 LM were significantly more sensitive (with decreased cell viability) to STAT3 inhibitors compared to either wt-MED12 LM or MyoF (FIG. 2A,B). Using the inventors' recently published CRISPR-engineered Gly44 mut-MED12 UtSM cell model⁵⁶, the inventors confirmed the preferential sensitivity of mut-MED12 compared to parental wt-MED12 UtSM cells to STAT3 inhibition (FIG. 2C,D). Together, these results indicate that mut-MED12 LM cells exhibit a heightened dependence on activated STAT3 signaling for growth.

### (2) STAT3-Activating Cytokines are Specifically Upregulated in MyoF Adjacent to Mut-MED12 LM.

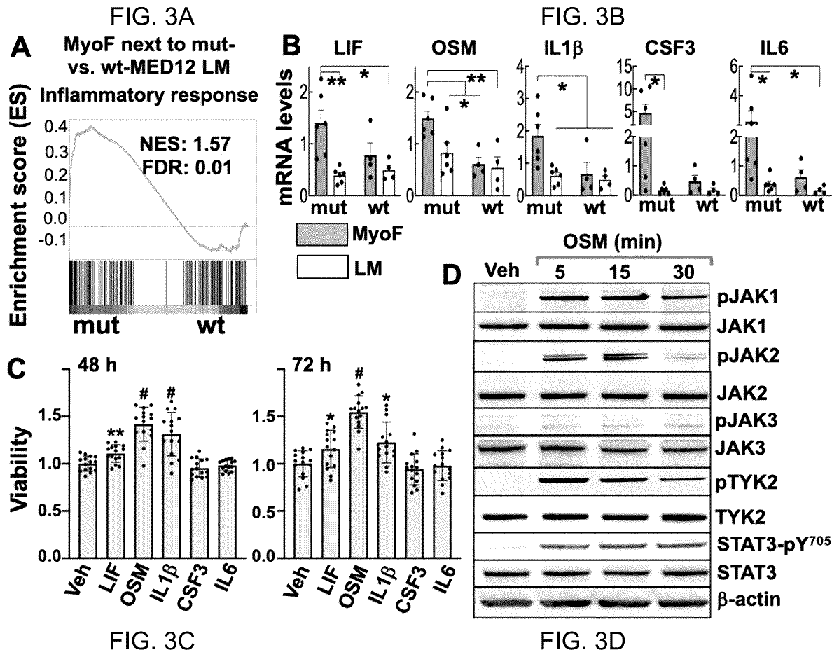

The inventors recently reported that MyoF located adjacent to mut-MED12 LM exhibits a distinct transcriptional profile compared to MyoF adjacent to wt-MED12 LM63. Gene set enrichment analysis (GSEA) revealed a significant enrichment of inflammatory pathways in MyoF adjacent to mut-MED12 LM (FIG. 3A); further inspection identified STAT3-activating cytokines, including OSM, LIF, IL1β, IL6, and CSF3, as significantly upregulated in these MyoF tissues (FIG. 3B) 47. Cytokines activate STAT3 primarily through JAK family proteins, a group of intracellular tyrosine kinases that include JAK1, JAK2, JAK3, and TYK247. The inventors found that treatment with individual cytokines OSM, LIF or IL1β significantly increased mut-MED12 LM cell viability, with OSM showing the strongest effect (FIG. 3C). OSM stimulated the phosphorylation of JAK1, JAK2, TYK2, and Y⁷⁰⁵-STAT3, but not JAK3, in mut-MED12 primary LM cells (FIG. 3D). Prior studies have shown that the region at the LM periphery is the most biologically active, with high levels of gene expression, vessel density, and proliferation, but a low level of hyaline degeneration, suggesting that local nutrition is important for LM growth⁶⁴. These findings suggest that cytokines secreted by MyoF potentially promote mut-MED12 LM growth by activating the JAK/STAT3 pathway.

### (3) STAT3 Activity Regulates the Expression of Genes Important for Cell Growth and ECM Production in Mut-MED12 LM.

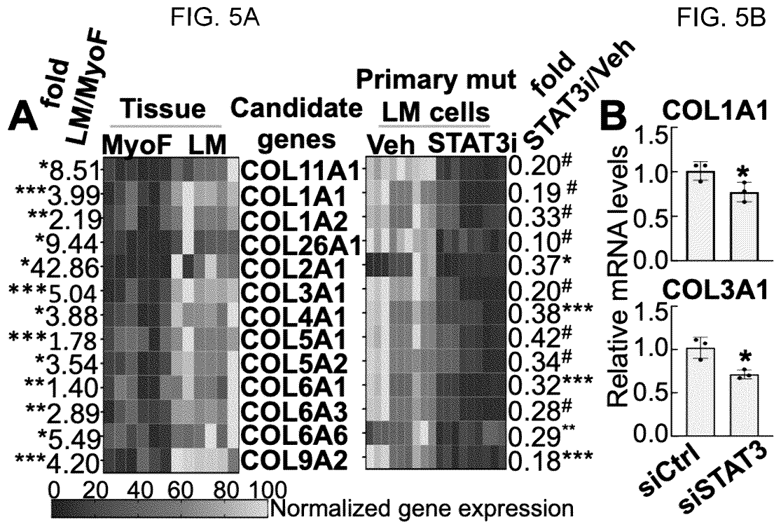

To determine the mechanism underlying STAT3-dependent growth of mut-MED12 LM cells, the inventors performed RNA-seq on mut-MED12 LM cells treated with vehicle (Veh) or the highly selective STAT3 inhibitor S3I-201. Gene Ontology (Panther) analysis identified “apoptosis signaling” to be the only pathway enriched by STAT3 inhibitor treatment (FIG. 4A,B), which was confirmed by GSEA analysis (FIG. 4C). Indeed, STAT3 inhibitor treatment of mut-MED12 LM cells led to significant upregulation of critical pro-apoptotic genes that were downregulated in mut-MED12 LM vs. MyoF in vivo based on the inventors' recently published LM and MyoF tissue RNA-seq data, including DDIT3 and BCL10 (FIG. 4D) 63. In contrast, STAT3 inhibitor downregulated the pathways “inflammation mediated by chemokine and cytokine signaling” and “WNT signaling,” which were shown to play important roles in LM growth⁶⁵⁻⁶⁸ STAT3 integrates common profibrotic pathways to promote tissue fibrosis^{47, 51, 52}, and over-production of ECM, especially collagen, is a hallmark of LM, especially the mut-MED12 subtype^{69, 70}. The inventors examined whether STAT3 inhibition downregulates collagen gene expression in mut-MED12 LM cells. STAT3 inhibitor decreased the expression of 13 collagen genes that were significantly upregulated in mut-MED12 LM vs. MyoF in vivo (FIG. 5A) 63. The inventors validated STAT3 inhibitor-mediated downregulation of COL1A1 and COL3A1 expression by STAT3 knockdown in mut-MED12 LM cells (FIG. 5B). These findings reveal STAT3 to be a key suppressor of pro-apoptotic genes and an activator of collagen genes, contributing to both LM growth and fibrosis.

### (4) CDK8/19 Activity is Required for the Expression of STAT3 Target Genes Important for Mut-MED12 LM Growth.

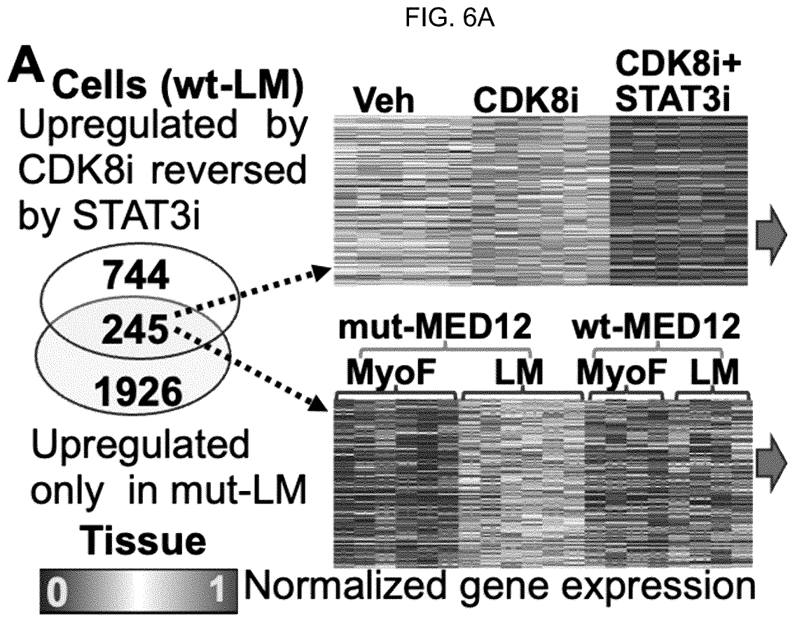

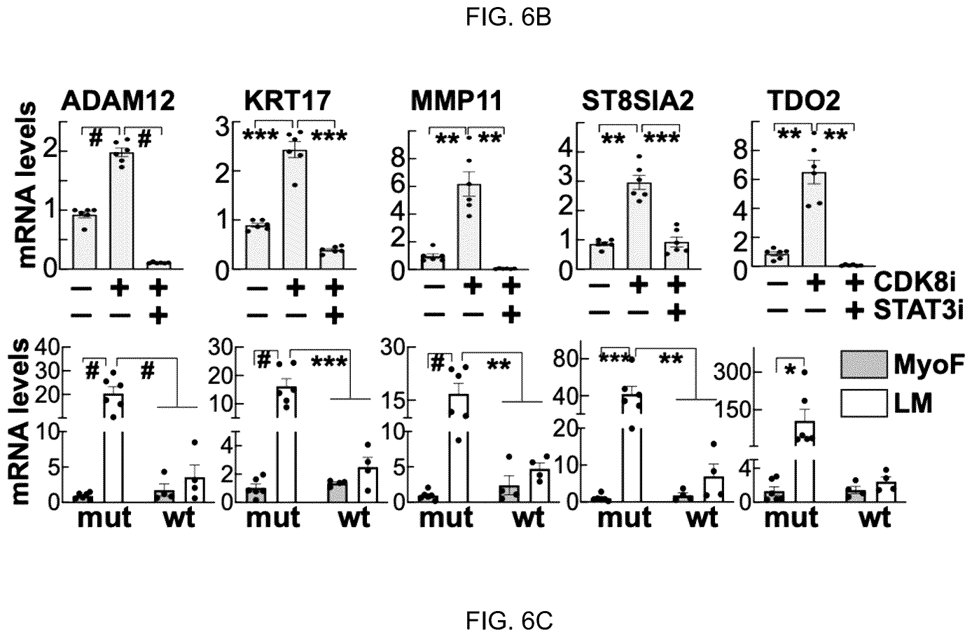

CDK8/19 activity, which the inventors have shown is disrupted by LM driver mutations in MED12, was also shown to negatively regulate STAT3-dependent transcription³⁶. The inventors investigated the contribution of CDK8/19 to STAT3-driven transcription in mut-MED12 LM. The inventors first performed RNA-seq to assess global gene expression changes in wt-MED12 LM cells treated with the highly specific CDK8/19 inhibitor SEL120 in the absence or presence of the STAT3 inhibitor S3I-201. This revealed that 989 genes were significantly upregulated by CDK8/19 inhibitor but reversed by STAT3 inhibitor co-treatment (FIG. 6A). The inventors filtered these CDK8/19/STAT3 co-regulated genes (n=989) against a set of genes (n=2171) found to be significantly upregulated in mut-MED12 LM compared to wt-MED12 LM and matched MyoF (FIG. 6A)⁶³. This analysis revealed an overlapping set of 245 CDK8/19 activity-dependent STAT3 target genes that are also uniquely expressed in mut-MED12 LM (FIG. 6A). The top-ranked genes (ADAM12, KRT17, MMP11, TDO2, ST8SIA2) have established roles in LM growth and fibrosis and were verified by real-time PCR (RT-qPCR) (FIG. 6B,C)^{63, 71-81}.

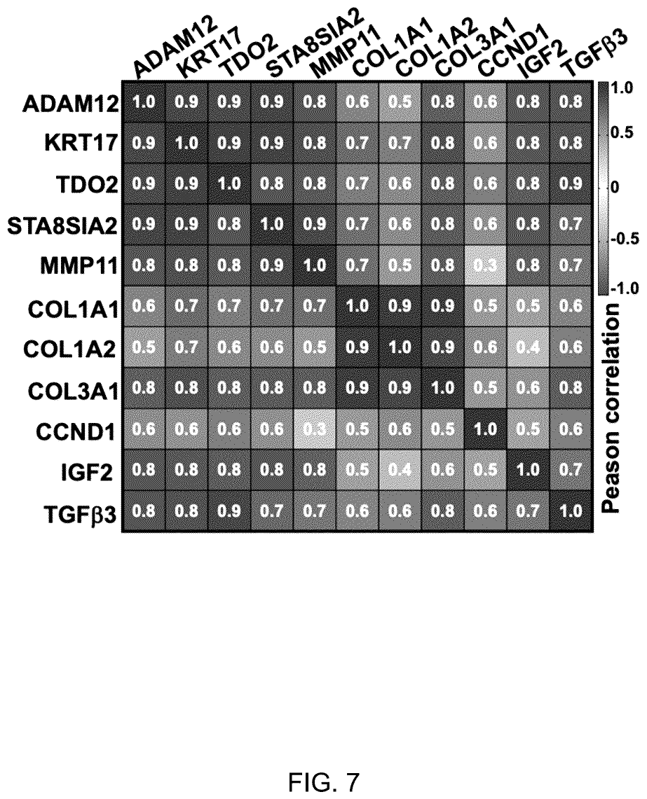

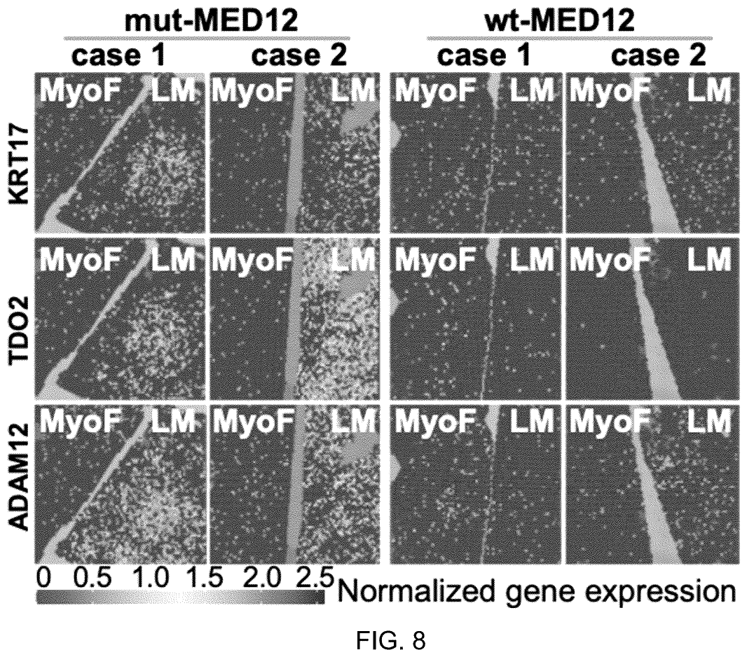

To determine the functional significance of these CDK8/19-regulated STAT3 target genes in LM growth in vivo, the inventors first determined the degree to which the mRNA levels of ADAM12, KRT17, MMP11, TDO2, and ST8SIA2 correlate to those of a gene subset with established roles in cell proliferation and LM growth (including COL1A1, COL1A2, COL3A1, CCND1, IGF2, and TGFβ3)^{70, 82, 83}. Pearson correlation analysis revealed a positive and highly significant correlation among these genes (FIG. 7). Next, the inventors performed spatial transcriptomics using paraffin-embedded tissues from mut-MED12 LM, wt-MED12 LM, and matched MyoF. This analysis verified that TDO2, ADAM12, and KRT17 were overexpressed only in mut-MED12 LM and were expressed in clusters matching the clonal growth pattern of mut-MED12 LM cells in vivo (FIG. 8).

### (5) STAT3 is Uniquely Required Among STAT Family Members for ADAM12 Expression.

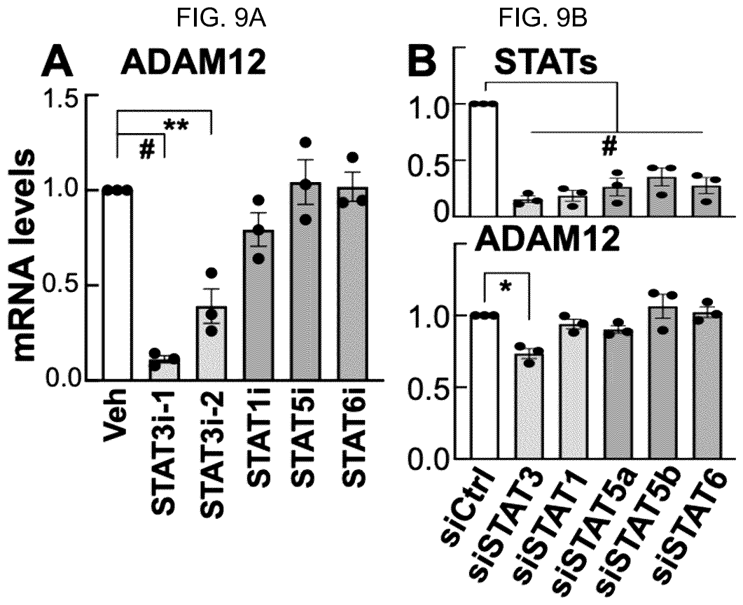

The STAT protein family consists of 7 members: STAT1, STAT2, STAT3, STAT4, STAT5A, STAT5B, and STAT6. While individual STATs recognize the same DNA binding site (GAS motif) and share certain functional roles, they are not entirely redundant and may be subject to unique regulation 84-88. ADAM12 is a key gene in the pathogenesis of various tumors and fibrosis⁷³⁻⁷⁵, and is also uniquely regulated by CDK8/19 loss and STAT3 in LM (FIG. 6). The inventors established that STAT3, but not other STATs, is uniquely required for the regulation of ADAM12 that is upregulated by CDK8/19 inhibition. The inventors found that in primary mut-MED12 LM cells, two STAT3 specific inhibitors, S3I-201 and C188-9, but not inhibitors of STAT1, STAT5, or STAT6, significantly downregulated ADAM12 expression (FIG. 9A). Furthermore, knockdown of STAT3, but not of STAT1, STAT5, or STAT6, also significantly reduced ADAM12 expression in mut-MED12 LM cells (FIG. 9B). The inventors' findings suggest that STAT3 is unique for mediating mut-MED12 LM growth mediated by CDK8/19 deficiency.

### (6) STAT3 DNA Binding and Transcriptional Activity is Higher in Mut-MED12 Compared to Wt-MED12 Cells.

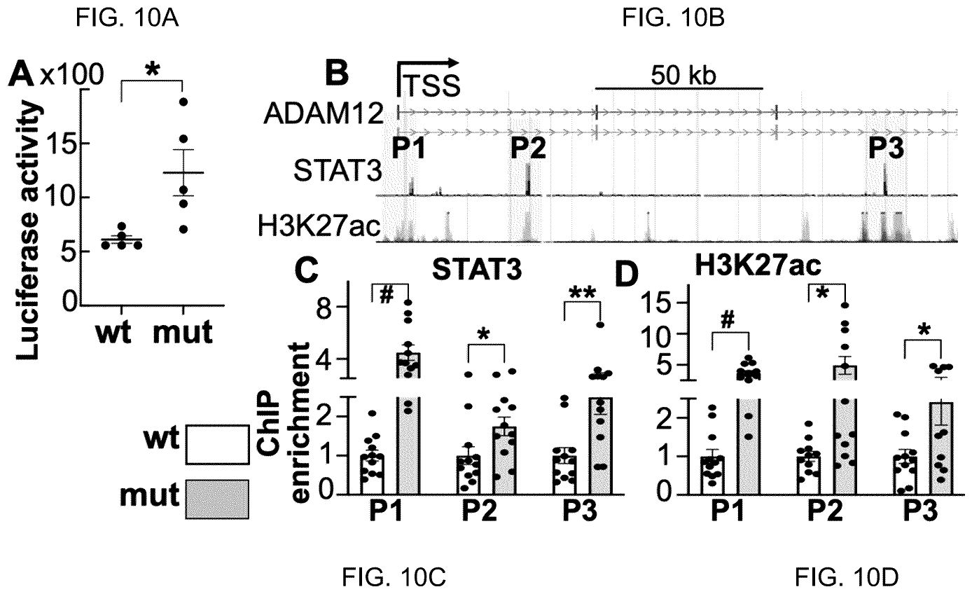

CDK8/19 activity was shown to regulate STAT3 transcriptional activity by controlling STAT3-chromatin resident time³⁶. The inventors investigated whether STAT3 transcriptional and DNA-binding activities were altered in mut-vs. wt-MED12 LM cells. First, using luciferase reporter assays, the inventors found that STAT3 transcriptional activity was significantly higher in mut-vs. wt-MED12 UtSM cells (FIG. 10A). Second, using ChIP-qPCR, the inventors found that STAT3 binding at multiple sites along the ADAM12 gene locus was significantly higher in mut-vs. wt-MED12 LM tissue, which was accompanied by enhanced presence of the active histone mark H3K27ac at these sites (FIG. 10B-D). These findings are consistent with upregulated ADAM12 expression in mut-MED12 LM (FIG. 6, 8) and support a mechanistic link between impaired Mediator kinase activity and hyperactivated STAT3 signaling in mut-MED12 LM.

(7) Mut-MED12-Induced Mediator Kinase Disruption Decreases pS⁷²⁷-STAT3 Levels but Increases pY¹⁰⁵-STAT3 Levels.

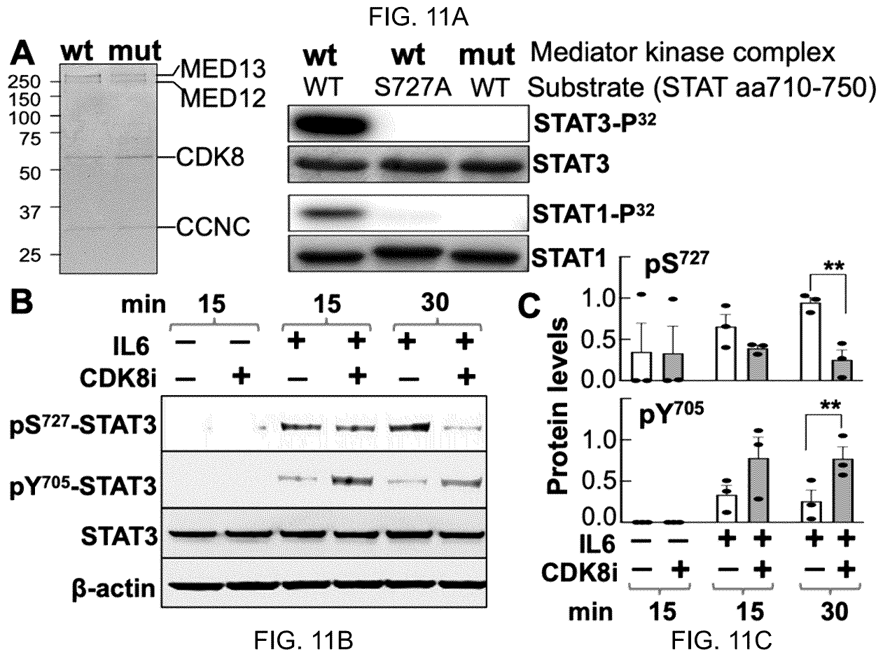

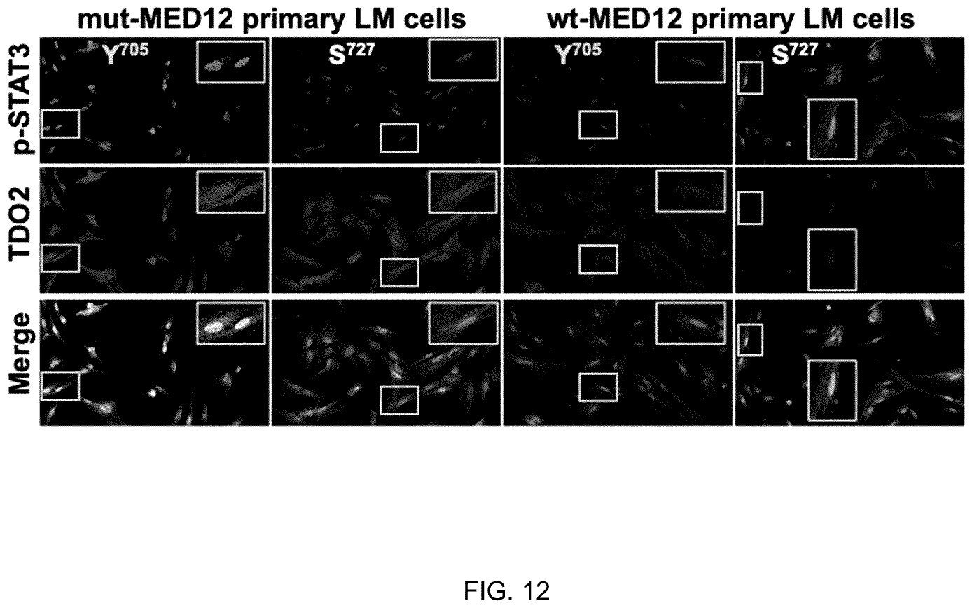

Prior work has shown that CDK8 (or other kinases) can phosphorylate S⁷²⁷-STAT3, which stimulates dephosphorylation of pY⁷⁰⁵-STAT3, converting the latter from a transcriptionally active to an inactive phospho-species^{36, 41-44, 47}. The inventors investigated whether mut-MED12-induced CDK8/19 impairment affects STAT3 phosphorylation at S⁷²⁷ and Y⁷⁰⁵. First, using in vitro kinase assays reconstituted from intact 4-subunit Mediator kinase modules carrying wt- or mut (G44D)-MED12, the inventors found that S⁷²⁷ of STAT3 was phosphorylated only by wt- but not by mut-MED12-bearing kinase modules (FIG. 11A). As expected, STAT1 phosphorylation at S⁷²⁷, an established biomarker of CDK8/19 activity^{37, 38, 40}, was also phosphorylated only by wt- but not by mut-MED12-bearing kinase modules (FIG. 11A). Second, treatment with CDK8/19 inhibitor SEL120 significantly decreased the level of pS⁷²⁷-STAT3 but increased that of pY⁷⁰⁵-STAT3 induced by IL6 in primary wt-MED12 LM cells (FIG. 11B,C).

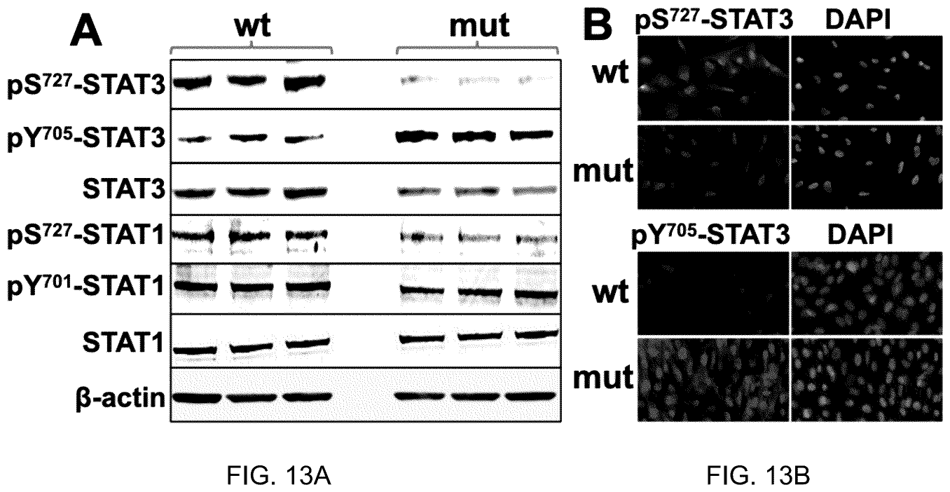

The inventors' previous and current studies identified TDO2 as a CDK8/19-dependent STAT3-target gene, which was upregulated in mut-MED12 LM and required for mut-MED12 LM growth (FIG. 6B,C)^{56, 63, 80, 81}. The inventors performed double immunostaining using antibodies specific for TDO2 and either pY⁷⁰⁵-STAT3 or pS⁷²⁷-STAT3 in mut- and wt-MED12 primary LM cells (FIG. 12). Confocal microscopy revealed that mut-MED12 primary LM cells express high levels of TDO2 and pY⁷⁰⁵-STAT3, but low levels of pS⁷²⁷-STAT3, whereas wt-MED12 cells express high levels of pS⁷²⁷-STAT3 only (FIG. 12). This further supports a role for dysregulated STAT3 activity as a function of Mediator kinase disruption in mut-MED12-driven LM growth. The inventors also observed the antagonistic effect of pS⁷²⁷-STAT3 on pY⁷⁰⁵-STAT3 using the inventors' CRISPR-generated Gly44 mut-MED12 UtSM cell line; the inventors found that the level of pS⁷²⁷-STAT3 was significantly lower but the level of pY⁷⁰⁵-STAT3 was markedly higher in mut-vs. wt-MED12 UtSM cells (FIG. 13). The inventors observed a significantly lower level of pS⁷²⁷-STAT1 in mut-MED12 UtSM cells, validating the impairment of CDK8/19 activity (FIG. 13A). Notably, the level of pY⁷⁰¹-STAT1 was similar between mut- and wt-MED12 UtSM cells (FIG. 13A), revealing the impact of pS⁷²⁷-STAT3 on pY⁷⁰⁵-STAT3 to be specific in the context of mut-MED12-induced CDK8/19 disruption in LM cells. Taken together, these data indicate that STAT3 is subject to an antagonistic phosphorylation shift between S⁷²⁷ and Y⁷⁰⁵ in wt-vs. mut-MED12 LM cells as a function of MED12-regulated CDK8/19 activity. Importantly, because the ratio of these two phospho-species determines the relative transcriptional activity of STAT3 (with a high ratio of pY⁷⁰⁵/S⁷²⁷-STAT3 being transcriptionally active) 41, 43, 47, the inventors predict that STAT3 is hyperactivated and drives growth of mut-MED12 LM as a consequence of impaired Mediator kinase activity, fully consistent with the enhanced transcriptional and DNA-binding activities observed in mut-MED12 LM (FIG. 10).

### (8) Progestins and PGR are Important for STAT3 Pathway Function.

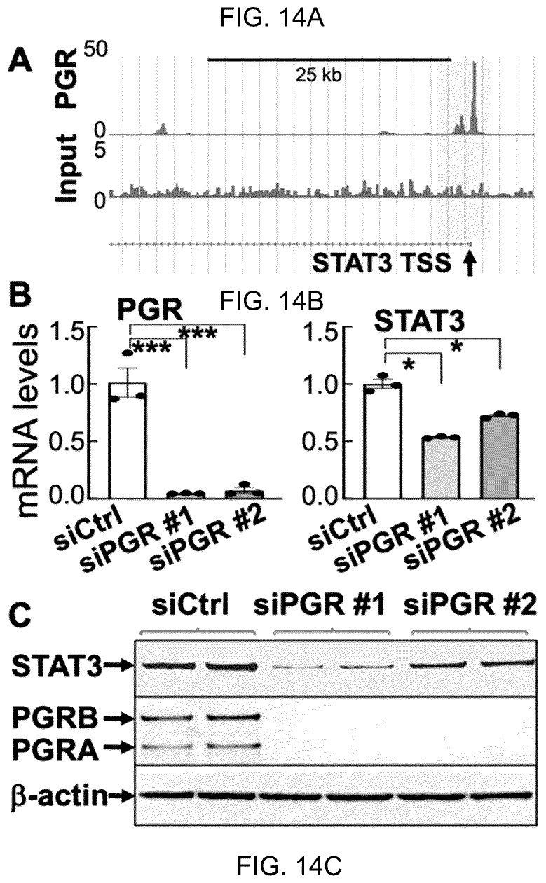

P4 acting through PGR is essential for LM growth¹³; thus, the inventors examined a possible link between PGR signaling and STAT3. In alignment with previous reports in breast cancer cells that progestin upregulates STAT3 protein levels in a PGR-dependent manner⁸⁹, the inventors found that PGR was enriched in the STAT3 gene promoter in LM tissue, and PGR knockdown in LM cells decreased STAT3 mRNA and protein levels (FIG. 14). Moreover, treatment of breast cancer cells with a progestin was reported to induce nuclear translocation of STAT3, its association with PGR and STAT3 transcriptional activity⁹⁰. These findings suggest that P4 action is important for STAT3 function.

### (9) MED12 Mutation Activated STAT3 Pathway as a Therapeutic Target in LM.

The inventors' preliminary findings identify STAT3 as a novel signaling hub at which MED12 mutation-induced Mediator kinase disruption and steroid hormone action converge, rendering STAT3 as a novel therapeutic target in LM, particularly the dominant mut-MED12 LM subtype. The inventors hypothesize that MED12 mutation-induced Mediator kinase disruption alters the phosphorylation pattern of STAT3 to favor a hyperactivated form, one with enhanced chromatin binding properties leading to transcriptional activation of a gene expression program that drives LM growth. The inventors propose that STAT3 inhibition using FDA-approved or in-pipeline agents targeting the JAK/STAT3 pathway will block ovarian steroid-dependent growth of the dominant mut-MED12 subtype (see FIG. 1 in Specific Aims).

### 2. Experimental Design

### Model Systems.

The inventors will use 3 model systems: (i) primary (passage 0 or un-passaged) human MyoF and LM cells genotyped for MED12 mutations, (ii) mice carrying xenografts of human primary MyoF and LM cells for mechanistic in vivo experiments, and (iii) human MyoF and LM tissues to verify the molecular markers and cell growth activities in vivo. The inventors used these model systems for the past 20 years to make numerous discoveries regarding mechanisms driving LM tumorigenesis, including the identification of LM stem cells and the role of P4 action^{13, 66, 91-94}. Because repeated passages in vitro cause depletion of mut-MED12 LM cells⁹⁵, the inventors optimized the culture conditions using Lonza Smooth Muscle Cell Growth Medium and high seeding cell density and found that the first passage cells maintain their MED12 mutation status and candidate gene expression patterns comparable to the original tissues^{63, 79}. Although the inventors' CRISPR-engineered mut-MED12 UtSM cell lines recapitulate crucial mut-MED12 LM-associated cellular, transcriptional, and metabolic alterations⁵⁶, the inventors generated the majority of the inventors' preliminary data using the primary wt- or mut-MED12 LM cells. Thus, the inventors propose xenograft experiments employing the new mut-MED12 UtSM cell lines as an alternative strategy to address the potential variability associated with patient samples.

### Scientific Rigor and Reproducibility and Relevant Biological Variables.

For Aims 1 and 2A, the inventors will run triplicate measurements in each experiment and repeat all experiments multiple times using samples from different patients. Multiple scientists will perform the key experiments. To address sample heterogeneity, the inventors will use primary cells from 6 to 12 patients per experiment. Under 2B, the inventors will use clinical samples from 160 patients. The inventors will collect LM and matched MyoF tissues from premenopausal women who have not received hormone treatment at least within the last 3 months. The inventors will genotype each sample for MED12 mutations. All LM samples will be histologically verified and stratified by size, patient race/ethnicity, menstrual cycle stage, and age.

### AIM 1. Determine the Functional Role of the STAT3 Pathway in LM Growth In Vivo.

Aim 1A: Does Blocking the Function of STAT3 Reduce or Prevent the Growth of Mut-MED12 LM More Efficiently than Wt-MED12 LM in Vivo?

### Rationale:

It is well established that uterine LM-linked mut-MED12 destabilizes the CDK8/19 activation loop and decreases Mediator-associated kinase activity³¹⁻³⁵. However, the identity of key biological substrates downstream of CDK8/19 and how disruption of their phosphorylation as a consequence of LM driver mutations in MED12 contribute to tumor formation are unknown. The experimental results disclosed herein established that mut-MED12-induced Mediator kinase disruption alters the ratio of functionally antagonistic phosphorylation to one favoring STAT3 hyperactivation (FIG. 10-13). Under physiological conditions, STAT3 activation is rapid and transient, returning to the basal state within a short time¹⁰⁷. However, inappropriate hyperactivation of STAT3 plays a pivotal role in the initiation and advancement of diverse diseases, including cancer and fibrosis, through the promotion of cell proliferation and survival and the stimulation of ECM gene expression^{47, 50-54, 108, 109}. Enhanced activation of STAT3, caused by mut-MED12-mediated CDK8/19 disruption, promotes cell proliferation and survival, ECM production, and LM growth in vivo. Further, inhibition of STAT3 reduces or prevents this phenotype.

### Design:

The inventors' in vitro studies revealed that two highly specific STAT3 inhibitors, S3I-201 and C188-9, preferentially inhibited the viability of both mut-MED12 primary LM cells and CRISPR-engineered mut-MED12 UtSM cells compared to the corresponding wt-MED12 cells (FIG. 2). Here, the inventors will assess whether this holds true for tumor growth in vivo using the inventors' previously established, clinically relevant xenograft mouse model of human LM13. This model faithfully replicates the phenotypes of human LM in situ. In this model, human LM cells are xenografted under the kidney capsules of ovariectomized (OVX) immunocompromised (NSG, the NOD-scid IL2Rgnull) mice. As any experimental LM mouse model requires E2+P4 for the maintenance and growth of tumor tissue, all experiments will involve treatment with E2+P4, administered as slow-release hormone pellets implanted subcutaneously in OVX mice, as previously published^{13, 23}.

S3I-201 and C188-9, two STAT3 inhibitors used for the inventors' in vitro studies, are potent selective small molecule inhibitors designed to target the phosphotyrosyl peptide binding site within the STAT3 SH2 domain, preventing Y⁷⁰⁵ phosphorylation, dimerization, and activation^{58, 110} In vitro and in vivo studies demonstrate that C188-9 inhibits tumor growth and fibrosis by inducing apoptosis and inhibiting proliferation and ECM gene expression preferentially in cells containing persistently activated STAT3108,109. Toxicology studies in mice demonstrate that C188-9 is well tolerated and does not cause clinical, laboratory, or pathological abnormalities⁵⁸. C188-9 has shown target engagement, no toxicity, and evidence of clinical benefit in a Phase I study in patients with solid tumors⁶². Here, the inventors will test whether C188-9 more efficiently reduces or prevents the growth of mut-vs. wt-MED12 LM. Freshly isolated mut- or wt-MED12 LM cells will be mixed with collagen (1×10⁶ cells per collagen pellet) and xenografted into both kidneys of OVX NSG mice using the procedures the inventors published before¹³. The mice will be randomized to the following groups: (i) E2+P4+Veh and (ii) E2+P4+C188-9. C188-9 (50 mg/kg) or Veh (DMSO) will be given daily via i.p. injection, as this dose has been reported to inhibit skin and lung fibrosis in vivo^{108, 109}. Since significant tumor growth has been observed 4 weeks after E2+P4 treatment⁸³, mice will be treated for 4 weeks. After treatment, mice will be sacrificed, and tumor volume measured. Cell proliferation (Ki67, PCNA, Cyclin D1) and apoptosis (TUNEL, total and cleaved PARP, DDIT3 and BCL10 [FIG. 4D]) will be assessed using immunohistochemistry (IHC) or immunoblot (IB) analyses. The inventors will perform Masson's trichrome staining and IB or IHC for COL1A1 and COL3A1 expression to assess fibrosis. STAT3 target gene expression (ADAM12, KRT17, and novel candidate targets identified in 2A) will be evaluated by RT-qPCR, IHC, or IB. The levels of pY⁷⁰⁵-STAT3, pS⁷²⁷-STAT3, and STAT3 nuclear localization in regenerated tumors will be assessed by IHC or IB to investigate whether STAT3 phosphorylation pattern is different between the regenerated wt- and mut-MED12 LM and verify that C188-9 indeed inhibits STAT3 activation in the inventors' mouse model in vivo.

### Aim 1B: Does OSM-Activated STAT3 Preferentially Enhance Mut-MED12 LM Growth in Vivo?

### Rationale:

The inventors' preliminary findings revealed that among the cytokines overproduced in MyoF adjacent to mut-MED12 LM, OSM vigorously activated the JAK/STAT3 pathway and showed the strongest pro-growth effect in mut-MED12 LM cells (FIG. 3). The observed viability induction by OSM was remarkable in primary mut-MED12 LM cells (FIG. 3). Increased levels of OSM was observed in tumor growth and fibrosis¹¹¹⁻¹¹³. OSM can promote fibroblast proliferation, providing a rationale for therapeutically targeting OSM-STAT3 signaling in fibrotic diseases, such as lung fibrosis^{112, 114-116}. The inventors hypothesize that OSM further enhances E2+P4-induced LM growth in vivo and to a greater extent in mice with mut-vs. wt-MED12 LM xenografts because OSM-activated STAT3 will remain transcriptionally active for longer periods in CDk8-deficient mut-MED12 LM cells.

### Design:

Freshly isolated mut- or wt-MED12 LM cells will be xenografted into OVX NSG mice, as described in Aim 1A. The experimental groups of each genotype (wt- or mut-MED12 LM cells) will be: (i) Veh, (ii) E2+P4, (iii) OSM, and (iv) E2+P4+OSM. Recombinant human OSM (25 μg/kg/day) or Veh (PBS) will be given via daily i.p. injection, as this dose was shown prior to be effective in vivo¹¹⁷. After 4 weeks, tumors will be harvested and evaluated as described in 1A.

### Aim 1C: Do JAK Family-Specific Inhibitors Reduce Tumor Growth Preferentially in Mut-MED12 LM?

### Rationale:

JAK family proteins, including JAK1, JAK2, JAK3, and TYK2, are the primary upstream activators of STAT347. The JAK/STAT3 pathway has long been known to play important roles in keloid pathogenesis, a process closely related to fibroid pathophysiology^{70, 118, 119}. Inhibitors of the JAK/STAT3 pathway have been shown to inhibit keloid progression via suppressing profibrotic gene expression and cell proliferation and stimulating apoptosis⁵². The inventors hypothesize that inhibition of JAK1/JAK2/TYK2 signaling will reduce LM growth in vivo and to a greater extent in mice bearing human mut-vs. wt-MED12 LM xenografts.

### Design:

The inventors showed that OSM treatment induced the phosphorylation of JAK1, JAK2, TYK2, but not JAK3 in mut-MED12 LM cells (FIG. 3D). Thus, the inventors will focus on JAK1, JAK2, and TYK2. The inventors will employ abrocitinib (JAK1 inhibitor), fedratinib (JAK2 inhibitor), and deucravacitinib (TYK2 inhibitor), FDA-approved oral drugs for safely treating dermatitis, myelofibrosis, and plaque psoriasis, respectivelyl²⁰⁻¹²². The inventors will also include delgocitinib, an oral selective Pan-JAK inhibitor, that was recently approved in Japan for the treatment of atopic dermatitis¹²³. Freshly isolated mut- or wt-MED12 LM cells will be xenografted into OVX NSG mice, which will be randomized into the following groups: (i) E2+P4+OSM, (ii) E2+P4+OSM+JAK family inhibitor. Abrocitinib (7.5 mg/kg), fedratinib (100 mg/kg), deucravacitinib (15 mg/kg), or (delgocitinib 1 mg/kg) will be given orally once a day for 4 weeks as previously described¹²⁴⁻¹²⁷. Tumors will be harvested and evaluated as described in 1A. The inventors will also measure the phosphorylation levels of JAK1, JAK2, and TYK2 to confirm specific inhibition of each target protein.

To validate the specificity of the effects of the inhibitors on LM growth in vivo, the inventors will infect primary mut- or wt-MED12 LM cells (passage 0) with lentivirus expressing shRNAs specific for JAK1, JAK2, TYK2, or STAT3, using scrambled shRNA as control. Infected cells will be mixed with collagen and xenografted in OVX NSG mice supplied with E2+P4+OSM for 4 weeks, followed by tumor harvest and evaluation, as described in 1A. In addition, the inventors will assess the levels of STAT3, JAK1, JAK2, or TYK2 in harvested xenografts to confirm their depletion by corresponding shRNA. The inventors will also perform IHC for pY⁷⁰⁵-STAT3 to confirm that knockdown of JAK family members inhibits STAT3 activation in LM xenografts. Reduction of LM growth via genetic depletion of STAT3 and JAK family members in LM cells will provide rigorous evidence for the role of the JAK/STAT3 pathway in LM growth.

### Aim 1D: Does Knockdown of CDK8/19-Regulated STAT3-Target Genes, ADAM12, KRT17, MMP11, or ST8SIA2, Preferentially Inhibit Mut-MED12 LM Growth?

### Rationale:

Among the top-ranked CDK8/19 and STAT3 coregulated genes that are specifically overexpressed in mut-MED12 LM (ADAM12, KRT17, MMP11, TDO2, and ST8SIA2; FIG. 6B,C), TDO2 has recently been reported to play an important role in LM growth and its inhibition reduced LM growth in vivo in xenograft mouse model^{63, 79-81}. The products of the remaining 4 genes play roles in promoting tumor growth and fibrosis in other tissues⁷¹⁻⁷⁸. For example, ADAM12, also known as meltrin α, is a multifunctional zinc-dependent enzyme that sheds membrane-anchored proteins and thus regulates the availability of bioreactive molecules, such as cell-surface receptors, growth factors, and cytokines^{128, 129}. ADAM12 functions as a core fibrotic pathway across various fibrotic diseases^{72, 73}. Its upregulation in cancer correlates with poorer patient survival, as it facilitates cancer cell proliferation and survival^{75, 130-132}. Therefore, ADAM12 represents an attractive therapeutic target in cancer and fibrotic diseases. However, the function of ADAM12 and the other 3 STAT3-target gene products (KRT17, MMP11, and ST8SIA2) have not previously been studied in LM. The inventors hypothesize that genetic depletion of ADAM12, KRT17, MMP11, or ST8SIA2 inhibits the growth of LM in vivo and to a greater extent in mice bearing mut-vs. wt-MED12 LM xenografts.

### Design:

Primary mut- or wt-MED12 LM cells (passage 0) will be infected with lentivirus expressing shRNA specific for ADAM12, KRT17, MMP11, or ST8SIA2, using scrambled shRNA as control. Infected cells will be xenografted into mice supplied with E2+P4+OSM for 4 weeks, followed by tumor harvest and evaluation, as described in 1A. The inventors will quantify expression levels of ADAM12, KRT17, MMP11, or ST8SIA2 in the xenografts to confirm the depletion of target protein by their corresponding shRNA. Although treatment with a TDO2 inhibitor alone effectively inhibits LM growth in vivo⁸¹, knocking down a single gene may not be adequate to block LM growth. In such instances, the inventors will concurrently knock down multiple genes using a vector expressing multiple shRNAs (VectorBuilder). Reduction of LM growth (and to a greater extent in mut-vs. wt-MED12 LM xenografts) by depletion of these STAT3-target genes will further validate the critical role of STAT3 pathway in LM growth.

For each experiment proposed in Aim 1, power analyses based on the inventors' previous experiments with the LM xenograft model indicate that LM cells from 4 to 6 patients with a minimum of 4 mice per treatment group/patient sample will be needed to reach statistical difference between groups^{13, 92, 93, 133} The inventors will double the sample size to ensure safe margins.

### AIM 1. Expected Outcomes:

The inventors have successfully demonstrated the tumor-suppressive effects of various compounds, such as RU486 (a P4 antagonist), 5-Aza (a DNA methylation inhibitor), and RANK-Fc (a RANKL/RANK pathway inhibitor), in LM growth using the xenograft mouse model^{92, 133}. The inventors will continue to use this model for the proposed experiments. The inventors found that OSM robustly activated JAK/STAT3 pathway and significantly increased cell viability in non-malignant mut-MED12 LM cells (FIG. 3C,D). The inventors expect OSM treatment to enhance E2+P4-induced LM growth, particularly in mut-MED 12 xenografts; but the effect of OSM alone may be mild, given the stimulative role of P4/PGR in STAT3 expression (FIG. 14). The inventors also expect that treatment with JAK/STAT3 pathway inhibitors or shRNA-mediated depletion of STAT3 or JAK family proteins will reduce tumor size more significantly in mut-vs. wt-MED12 xenografts, with the mut-MED12 LM xenografts showing lower proliferative and higher apoptotic indices and producing less ECM. The pan-JAK inhibitor may exhibit stronger inhibitory effects than single JAK inhibitors. In addition to being activated by cytokine receptor-activated JAK proteins, STAT3 can also be activated by non-receptor (such as Bcr-Abl and Src) or receptor (such as EGFR and PDGFR) tyrosine kinases^{47, 50, 51}. Therefore, the inventors anticipate a stronger effect of the STAT3 inhibitor on reducing LM growth compared to inhibitors targeting JAK family proteins. The inventors expect to detect higher pY⁷⁰⁵-STAT3 levels but lower pS⁷²⁷-STAT3 levels in mut-MED12/CDK8/19-deficient vs. wt-MED12 LM xenografts treated with OSM plus or minus E2+P4. The inventors also anticipate that pY⁷⁰⁵-STAT3 levels will be significantly downregulated in mut-MED12 LM xenografts when the JAK/STAT3 pathway is blocked pharmaceutically or using shRNAs.

### AIM 1. Potential Pitfalls/Alternative Approaches:

In addition to the JAK/STAT3 pathway, cytokines may also regulate cell proliferation and death via other pathways, such as the MAPK and PI3K/AKT pathways¹³⁷; thus, the inventors will perform IHC of phosphorylated ERK and AKT in xenografts derived from mice treated with OSM to assess potential activation of these pathways in vivo. Among the 7 STAT family proteins, the transcriptional activity of STAT1, STAT3, and STAT5 has been reported to be modified by Mediator kinase³⁶⁻⁴⁰. In most cells, these STAT proteins are activated by different mechanisms and bind to distinct loci to regulate specific target gene expression; however, they can also bind to the same regulatory loci resulting in compensatory or antagonistic signaling⁸⁴⁻⁸⁸. Nonetheless, the inventors' findings that the pY⁷⁰¹. STAT1 level was not increased in mut-MED12 cells (FIG. 13A) and, further, that ADAM12 gene expression was specifically suppressed by inhibiting STAT3 but not other STAT proteins (FIG. 9) suggest a specific role for Mediator kinase disruption in transcriptionally activating a STAT3-dependent gene expression program that drives LM cell growth.

AIM 2. Define the Cell Populations with Unique Epigenetic and Transcriptomic Processes Responsible for STAT3-Driven Growth in Mut-MED12 LM and Validate the Mechanistic Findings in Clinical Tissue Samples.
Aim 2A: What are the STAT3 Activity-Dependent Gene Signatures Dysregulated Specifically in Mut-MED12 LM Cells and their Underlying Epigenetic Mechanisms at the Single-Cell Level?

### Rationale:

Upon activation by cytokines, STAT3 binds to gene promoters or enhancer elements, where it engages with diverse transcriptional regulators and chromatin remodeling proteins, orchestrating intricate effects on the epigenomic and transcriptomic landscape of target genes¹³⁸. The inventors identified a subset of 245 CDK8/19 activity-dependent STAT3 target genes that are also uniquely expressed in vivo in mut-MED12 LM vs. wt-MED12 LM and matched MyoF tissues (FIG. 6). Intriguingly, the cells expressing these genes (e.g., ADAM12, KRT17, TDO2) exist in mut-MED12 LM tissue in vivo in a distinct clonal growth pattern (FIG. 8), which suggests that they are neoplastic smooth muscle cells with mut-MED12 and play important biological roles in tumorigenesis. Previous studies reported that the mut-MED12 LM tissue contains substantial numbers of tumor-associated fibroblasts that do not carry a mutation, and the ratio of neoplastic smooth muscle cells with somatic mutations to non-mutated cell types (e.g. fibroblasts and endothelial and immune cells) vary between different LM subtypes driven by different mutations^{69, 98}. This cellular heterogeneity may account for the divergent responses of different mutation-driven LMs, such as mut-MED12 and HMGA2-overexpressing tumors, to antiprogestin or other treatments¹³⁹⁻¹⁴². Thus, it is important to assess cell-specific differences in mRNA expression or chromatin accessibility in various cell populations of mut-MED12 LM and their response to STAT3 inhibition.

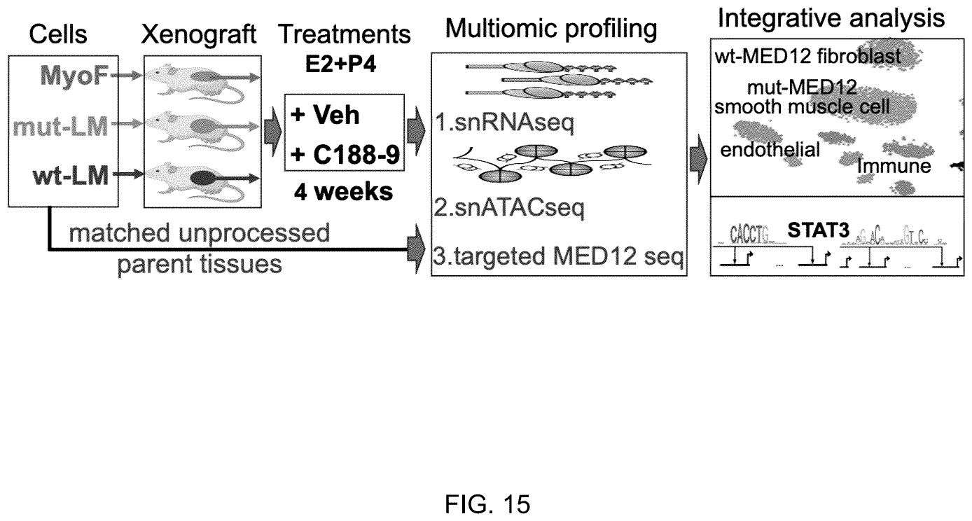

Additionally, multiple bulk RNA-seq studies of LMs with different driver gene mutations and matched MyoF have reported that mut-MED12 LM demonstrate distinct epigenetic signatures and transcriptional profiles^{24, 63, 143-145}. While these bulk-level genome-wide studies shed some light on the biological processes in mut-MED12-driven LM, the fundamental question-what epigenetic mechanisms account for the specific transcriptional aberration in mut-MED12 cells remains unanswered. The single-nucleus multiomics technique, which simultaneously profile gene expression (snRNA-seq), chromosome accessibility (snATAC-seq), and MED12 mutation status (MED12-targeted sequencing) in the same single cell, can unprecedentedly advance the inventors' knowledge of cell subtype-specific cis-trans interactions between regulatory elements and transcription factors, and the genes dysregulated by these networks that contribute to disease development in mutated or unmutated cells. Thus, to test the hypothesis that mut-MED12-induced loss of CDK8/19 activity alters the STAT3-associated epigenetic signature, which drives the expression of a distinct set of genes crucial for LM cell proliferation, survival, and ECM production in a cell subtype-specific manner in mut-MED12 LM, the inventors will perform parallel snRNA-seq, snATAC-seq and MED12-targeted long-read sequencing in individual cells isolated from matched patient MyoF and LM expressing wt-MED12 or mut-MED12 tissues and corresponding tissue-derived xenografts treated with E2+P4 with or without STAT3 inhibitor C188-9 (FIG. 15). A deeper understanding of the epigenetic mechanisms controlling distinct gene expression programs in neoplastic cells with mut-MED12 and other cell populations is prerequisite to successfully identifying specific biomarkers and molecular targets for therapeutics.

### Design:

Freshly isolated cells from mut-MED12 LM, wt-MED12 LM and MyoF will be xenografted into OVX NSG mice, which will be randomized to two treatment groups for each tissue type: (i) E2+P4+Veh and (ii) E2+P4+C188-9 (FIG. 15). After 4 weeks of treatment, the regenerated xenografts and the parent tissues, from which they originated, will be subjected to nuclei isolation using Chromium Nuclei Isolation Kit (10× Genomics). The isolated nuclei will be submitted to a core facility for library preparation and sequencing. Libraries (snRNA-seq and snATAC-seq) will be generated using the Chromium Next GEM Single Cell Multiome ATAC+Gene Expression Reagent Kit and will be sequenced using Illumina NovaSeq X Plus with a depth of >30,000 reads/nuclei for each library. To identify the nuclei carrying MED12 mutation, full-length cDNA will be used to generate MED12 full-length transcript library, which will be used for the long-read sequencing (Oxford Nanopore). The MED12-targeted long-read sequencing results will be integrated with the parallel snRNA-seq/snATAC-seq data to understand the interactions between mut-MED12 and STAT3-related chromatin accessibility and mRNA expression in a cell-specific fashion. In the patient-derived xenograft mouse model, the inventors showed that MyoF or LM xenografts form tissues in response to E2+P4 treatment with the typical histology of original tissues¹³. Here, the inventors include unprocessed parent tissues as controls in the single-nucleus sequencing analyses to further validate that xenografted MyoF and LM tissues from mice treated with E2+P4 will behave as original tissues cellularly (e.g., neoplastic smooth muscle cells with mut-MED12 and tumor-associated fibroblast cells with wt-MED12) and molecularly (e.g., chromosome accessibility and transcriptome)⁶⁹, thereby establishing, at the single-cell level, the clinical relevance of using the xenograft mouse model to investigate the tumorigenic and therapeutic mechanisms of human uterine LM. Although MyoF xenografts do not grow significantly as LM xenografts, they survive and form myometrium-like tissues histologicallyl³. Therefore, the inventors expect to generate sufficient numbers of cells from MyoF xenografts for sn-seq studies. Power analysis based on the inventors' previous single-cell omics work showed that a minimum of 3 patient samples will be needed to show significant differences between different groups^{98, 146} The inventors will use 6 patients samples/treatment group to ensure safe margins.

### Integrated Bioinformatics Analyses:

Raw reads from snRNA-seq/snATAC-seq will be processed using Cell Ranger Arc (10× Genomics) with the human reference genome (GRCh38). Most cells in LM xenografts, including vascular cells, are of human origin and the mouse cells in the xenografts will be removed using XenoCell^{147, 148} snNanoGPS will be used to analyze the MED12-targeted sequencing data to discern nuclei with the MED12 gene mutation¹⁴⁹. Various tools in the Seurat Suite will be used to cluster snRNA-seq/snATAC-seq data generated on the original mut-MED12 LM, wt-MED12 LM, and MyoF tissues and their corresponding xenograft tissues¹⁵⁰⁻¹⁵², and each cluster will represent different cell subsets (as conceptualized in FIG. 15). Differentially expressed genes and differentially accessible regions between MED12-mutated and non-mutated cell clusters in response to STAT3 inhibitor treatment will be identified and overrepresented motifs will be detected. Putative regulatory targets will be identified by direct linkage of differentially accessible DNA regions to proximal differentially expressed genes in the same nucleus. Velocyto and Monocle 3 will be used to infer the developmental trajectories of cells^{153, 154}. Substantial information about the cell subsets can be extracted from the integrative analyses of the single-nucleus data. The inventors will ask the following questions. What is the potential evolutionary trajectory from MyoF cells to LM cells with respect to chromatin accessibility switch and gene expression program shift in response to E2+P4 in LM growth? What cell subsets, epigenic mechanisms, or gene expression programs are potentially responsible for the differential response to STAT3 inhibitor-induced growth restriction between mut- and wt-MED12 LM? What are the regulatory regions and potential transcription factors that specifically repress the expression of pro-growth or profibrotic genes in mut-MED12 cell populations in response to STAT3 inhibition? For genes specifically expressed in cell populations that carry MED12 mutations: is chromatin accessibility specifically high or STAT binding motif enriched in their loci or does STAT3 inhibitor treatment specifically inhibit their expression? To narrow down the genes coregulated by CDK8/19 and STAT3 activity, the inventors will integrate the snRNA-seq/snATAC-seq dataset with the RNA-seq data of CDK8/19 inhibitor treated primary wt-MED12 LM cells (FIG. 6). The promising target genes will be assessed using RT-qPCR, IB, and IHC to confirm their specific expression in mut-MED12 LM cells. Functional analysis of these genes by knockdown or small molecule inhibitors (if available) will be performed to determine their effects on mut-MED12 LM cell proliferation, apoptosis, and ECM production using cell culture and xenograft mouse models.

### Aim 2B: To Validate the Relationship Between STAT3 Pathway Activation and Proliferation, Apoptosis and ECM Production in Cell Populations of a Large Set of Mut-MED12 and Wt-MED12 LM Tissues In Vivo.

### Design:

To further establish the potential role of the hyperactive STAT3 pathway induced by mut-MED12-mediated loss of CDK8/19 activity in LM growth in vivo in humans, the inventors will perform IHC and RNAScope in paraffin-embedded or frozen mut-MED12 LM, wt-MED12 LM, and matched MyoF tissues. The inventors will assess cell-specific mRNA and protein levels of CDK8/19 activity-regulated STAT3 target genes (ADAM12, KRT17, TDO2, MMP11, ST8SIA2, and the top 10 novel genes identified in 2A), markers of cell proliferation (Cyclin D1, Ki67), apoptosis (TUNEL) and ECM production (COL1A1, COL3A1, trichrome stain), and cytokines IL1β, OSM, and LIF using IHC, RNAScope, and ImageJ analysis. The inventors will also assess pS⁷²⁷-STAT3 and pY⁷⁰⁵-STAT3 status of cells using phospho-specific antibodies. All tissues will be genotyped for MED12 mutation using Sanger sequencing. Based on the inventors' bulk RNA-seq and spatial transcriptome data in mut- and wt-MED12 LM and MyoF tissues (FIG. 8) 63, power analyses indicate that with 60-80 patient samples and an effect size of 0.8 and a of 0.05, the inventors will achieve a power of 0.8-0.9. Since MED12 mutations occur in 77.4% of all LMs, the inventors will recruit a total of 160 patients (approximately 36 samples will be wt-MED12 LM) to maximize the probability of obtaining meaningful results. Thus, over a period of 4 years, the inventors plan to collect a total of 40 LM samples per year. Annually, more than 300 hysterectomies are performed for uterine LM at Prentice Women's Hospital; thus, the inventors do not expect any difficulty in collecting a sufficient number of samples. To minimize sample variation, the inventors will follow the criteria described under Scientific Rigor and Biological Variables to collect samples (also see Human Subjects component). Given that various factors, such as the ex vivo delay time in tissue processing and the manner of tissue handling, influence stability of phosphoproteins in tissue following excision, the inventors will also follow the tissue procurement guidelines established previously for studying signal pathway phosphoproteins¹⁵⁵. The inventors will use 2-way ANOVA followed by Tukey multiple comparisons to compare the levels of the proposed markers between mut-MED12 LM, wt-MED12 LM, and matched MyoF tissues. Pearson's correlation will be performed to assess the correlation between the levels of pY⁷⁰⁵-STAT3, pS⁷²⁷-STAT3, STAT3 target gene expression, the markers of proliferation, apoptosis and ECM production, and cytokines. This translational study will complement the inventors' xenograft studies by directly validating the pathologic relevance to the STAT3 target gene signatures that are specifically dysregulated by mut-MED12-mediated disruption of CDK8/19 activity in a large and independent set of patient samples in vivo.

### AIM 2. Expected Outcomes:

snRNA-seq/snATAC-seq/MED12-targeted sequencing will provide a functional understanding of how the loss of CDK8/19 activity induced by mut-MED12, along with accessible chromatin region alterations, determines a STAT3 gene transcription program in mut-MED12 cells leading to LM growth. The inventors anticipate that single-nucleus multiome data analyses will divide cells into different clusters with each cluster representing a distinct cell type/subtype (e.g., smooth muscle cell cluster, fibroblast cell cluster) in mut-MED12, wt-MED12 LM, and MyoF tissues^{69, 98}. Within mut-MED12 LM, the inventors expect to identify a subset of genes with the highest expression level accompanied with the highest chromatin accessibility in the specific cell population expressing mut-MED12: (i) these genes will promote mut-MED12 cell proliferation, survival, or ECM production; (ii) they can also act in a paracrine manner to activate the surrounding unmutated cells; (iii) STAT3 inhibitor C188-9 treatment will block or decrease their expression (e.g., ADAM12, KRT17, TDO2 and newly identified genes); and (iv) they will highly overlap with the list of genes that are upregulated by CDK8/19 inhibitor but reversed by STAT3 inhibitor treatment in wt-MED12 primary LM cells (FIG. 6). C188-9 treatment will induce the expression of pro-apoptotic genes (e.g., DDIT3, BCL10, FIG. 4D) specifically in mut-MED12 LM cells. The motif analysis of differential open chromatin regions will identify critical and novel transcription factors that are key for cell proliferation, survival, and ECM production and eventually LM formation. The analysis will potentially allow us to identify specific markers that label mut-MED12 cells in LM tissue in vivo. The inventors will attempt identify a population of MED12-mutated tumor stem cells with refined markers. In a separate a large subset of clinical LM samples, the inventors expect to observe a significant positive correlation between the expression levels of the markers of cell proliferation, decreased apoptosis and ECM production and STAT3 target genes, and validate expression of a subset of genes identified under 2A. The inventors expect that smooth muscle cells in mut-MED12 LM tissue will contain higher levels of pY⁷⁰⁵-STAT3 and lower levels of pS⁷²⁷-STAT3 compared with those in wt-MED12 LM.

### AIM 2. Potential Pitfalls/Alternative Approaches:

Although limited data suggest that unphosphorylated STAT3 may dimerize and regulate gene expression¹⁵⁶, the inventors clearly demonstrated a unique gene signature upregulated by CDK8/19 inhibitor and downregulated by cotreatment with STAT3 inhibitor S³I-201 (FIG. 6) that specifically inhibits Y⁷⁰⁵ phosphorylation of STAT3 without affecting total STAT3 level¹¹⁰, suggesting that transcriptional activation by mut-MED12-induced CDK8/19 loss is dependent on pY⁷⁰⁵-STAT3. Various driver mutations of LM (MED12 vs HMGA2) introduce biological heterogeneity¹⁵⁷. The inventors will minimize this using a defined range of tumor size (3 to 8 cm), genotyping all tumor samples for MED12 mutations and HMGA2 overexpression and excluding samples from hormone-treated patients. The inventors may use frozen tissues and perform a series of double IHC staining to assess colocalization of the related markers. The inventors' CRISPR-engineered mut-MED12 UtSM cells, but not the parental cells with wt-MED12, regenerate LM-like lesions with ECM production in vivo and show STAT3-related abnormalities and biology identical to primary mut-MED12 LM cells (FIGS. 2&13)⁵⁶. If necessary, the inventors will use these cells as an alternative model to address the heterogeneity of patient samples.

OVERALL IMPACT AND FUTURE DIRECTIONS. The proposed studies will establish the role of STAT3 pathway hyperactivation, caused by mut-MED12-induced CDK8/19 activity loss in neoplastic cell proliferation, survival, ECM production, and eventually tumor growth. Importantly, the majority of the proposed studies will be performed using a preclinical primary LM cell xenograft mouse model, enhancing the physiological relevance of the study findings and providing a strong rationale for translational studies and drug development. The advancement of machine learning technology now makes it feasible to diagnose mut-MED12 LM noninvasively using MRI¹⁵⁸. The knowledge gained from the inventors' project could facilitate the development of novel non-hormonal therapeutics for LM, especially the mut-MED12 subtype, thus launching precision medicine for LM.

Future studies that remain outside the scope of this application will focus on further defining the molecular mechanisms, whereby CDK8/19-stimulated STAT3 S⁷²⁷ phosphorylation antagonizes Y⁷⁰⁵ phosphorylation and alters its transcriptional activity in mut-MED12 LM cells. Several proteins, such as phosphatase TC45 and PAS domain containing 1 (PASD1), were reported to interact with STAT3 and regulate its oncogenic transcriptional activity via controlling STAT3 phosphorylation at Y⁷⁰⁵, DNA-binding activity, and transcriptional activities^{43, 159, 160}. Additionally, the inventors successfully transformed the parent myometrial smooth muscle cell line UtSM to neoplastic LM cells via introducing the G44N/D MED12 mutations⁵⁶. In the future, the inventors will use CRISPR to introduce into these same cells mutations that disrupt the kinase functions of both CDK8 and CDK19 simultaneously to test whether these altered CDK8/19 kinase-dead cells will phenocopy the mut-MED12 UtSM cells. Also, the mut-MED12 cell populations with STAT3 hyperactivity in 2A will be isolated from LM tissues using the newly identified markers followed by STAT3 CUT&RUN-seq to define the STAT3 cistrome. The inventors will perform studies to understand the genome-wide interactions between activated STAT3 and liganded PGR in mut-MED12 LM.

Sequences
MED12 human AA (SEQ ID NO: 1)
Sequence

MAAFGILSYEHRPLKRPRLGPPDVYPQDPKQKEDELTALNVKQGFNNQPAVSGDEHGSAKNVS
FNPAKISSNFSSIIAEKLRCNTLPDTGRRKPQVNQKDNFWLVTARSQSAINTWFTDLAGTKPL
TQLAKKVPIFSKKEEVFGYLAKYTVPVMRAAWLIKMTCAYYAAISETKVKKRHVDPFMEWTQI
ITKYLWEQLQKMAEYYRPGPAGSGGCGSTIGPLPHDVEVAIRQWDYTEKLAMFMFQDGMLDRH
EFLTWVLECFEKIRPGEDELLKLLLPLLLRYSGEFVQSAYLSRRLAYFCTRRLALQLDGVSSH
SSHVISAQSTSTLPTTPAPQPPTSSTPSTPFSDLLMCPQHRPLVFGLSCILQTILLCCPSALV
WHYSLTDSRIKTGSPLDHLPIAPSNLPMPEGNSAFTQQVRAKLREIEQQIKERGQAVEVRWSF
DKCQEATAGFTIGRVLHTLEVLDSHSFERSDFSNSLDSLCNRIFGLGPSKDGHEISSDDDAVV
SLLCEWAVSCKRSGRHRAMVVAKLLEKRQAEIEAERCGESEAADEKGSIASGSLSAPSAPIFQ
DVLLQFLDTQAPMLTDPRSESERVEFFNLVLLFCELIRHDVFSHNMYTCTLISRGDLAFGAPG
PRPPSPFDDPADDPEHKEAEGSSSSKLEDPGLSESMDIDPSSSVLFEDMEKPDFSLFSPTMPC
EGKGSPSPEKPDVEKEVKPPPKEKIEGTLGVLYDQPRHVQYATHFPIPQEESCSHECNQRLVV
LFGVGKQRDDARHAIKKITKDILKVLNRKGTAETDQLAPIVPLNPGDLTFLGGEDGQKRRRNR
PEAFPTAEDIFAKFQHLSHYDQHQVTAQVSRNVLEQITSFALGMSYHLPLVQHVQFIFDLMEY
SLSISGLIDFAIQLLNELSVVEAELLLKSSDLVGSYTTSLCLCIVAVLRHYHACLILNQDQMA
QVFEGLCGVVKHGMNRSDGSSAERCILAYLYDLYTSCSHLKNKFGELFSDFCSKVKNTIYCNV
EPSESNMRWAPEFMIDTLENPAAHTFTYTGLGKSLSENPANRYSFVCNALMHVCVGHHDPDRV
NDIAILCAELTGYCKSLSAEWLGVLKALCCSSNNGTCGFNDLLCNVDVSDLSFHDSLATFVAI
LIARQCLLLEDLIRCAAIPSLLNAACSEQDSEPGARLICRILLHLFKTPQLNPCQSDGNKPTV
GIRSSCDRHLLAASQNRIVDGAVFAVLKAVFVLGDAELKGSGFTVTGGTEELPEEEGGGGSGG
RRQGGRNISVETASLDVYAKYVLRSICQQEWVGERCLKSLCEDSNDLQDPVLSSAQAQRLMQL
ICYPHRLLDNEDGENPQRQRIKRILQNLDQWTMRQSSLELQLMIKQTPNNEMNSLLENIAKAT
IEVFQQSAETGSSSGSTASNMPSSSKTKPVLSSLERSGVWLVAPLIAKLPTSVQGHVLKAAGE
ELEKGQHLGSSSRKERDRQKQKSMSLLSQQPFLSLVLTCLKGQDEQREGLLTSLYSQVHQIVN
NWRDDQYLDDCKPKQLMHEALKLRLNLVGGMFDTVQRSTQQTTEWAMLLLEIIISGTVDMQSN
NELFTTVLDMLSVLINGTLAADMSSISQGSMEENKRAYMNLAKKLQKELGERQSDSLEKVRQL
LPLPKQTRDVITCEPQGSLIDTKGNKIAGEDSIFKKEGLQVSTKQKISPWDLFEGLKPSAPLS
WGWFGTVRVDRRVARGEEQQRLLLYHTHLRPRPRAYYLEPLPLPPEDEEPPAPTLLEPEKKAP
EPPKTDKPGAAPPSTEERKKKSTKGKKRSQPATKTEDYGMGPGRSGPYGVTVPPDLLHHPNPG
SITHLNYRQGSIGLYTQNQPLPAGGPRVDPYRPVRLPMQKLPTRPTYPGVLPTTMTGVMGLEP
SSYKTSVYRQQQPAVPQGQRLRQQLQQSQGMLGQSSVHQMTPSSSYGLQTSQGYTPYVSHVGL
QQHTGPAGTMVPPSYSSQPYQSTHPSTNPTLVDPTRHLQQRPSGYVHQQAPTYGHGLTSTQRF
SHQTLQQTPMISTMTPMSAQGVQAGVRSTAILPEQQQQQQQQQQQQQQQQQQQQQQQQQQYHI
RQQQQQQILRQQQQQQQQQQQQQQQQQQQQQQQQQQHQQQQQQQAAPPQPQPQSQPQFQRQGL
QQTQQQQQTAALVRQLQQQLSNTQPQPSTNIFGRY

1. A method of treating uterine leiomyomas (LMs) in a subject in need thereof, the method comprising administering a therapeutically effective amount of a signal transducer and activator of transcription 3 (STAT3) pathway inhibitor to the subject to treat the LMs in the subject.

2. The method of claim 1, wherein the STAT3 pathway inhibitor comprises a STAT3 specific inhibitor or a Janus kinase (JAK) inhibitor.

3. The method of claim 2, wherein the STAT3 specific inhibitor is S3I-201, C188-9, WP1066, or VVD-130850.

4. The method of claim 2, wherein the JAK inhibitor is selected from abrocitinib, fedratinib, deucravacitinib, delgocitinib, baricitinib, upadacitinib, ruxolitinib, tofacitinib, and filgotinib.

5. The method of claim 1, wherein the LMs in the subject comprise at least one mutation in mediator complex subunit 12 (MED12).

6. The method of claim 5, wherein the mutation in MED12 comprises a mutation that alters MED12 function and inactivates CDK8/19 kinase activity.

7. The method of claim 6, wherein the mutation in MED12 comprises a mutation in G44, as compared to SEQ ID NO: 1.

8. The method of claim 7, wherein the mutation in G44 is a G44D substitution mutation.

9. The method of claim 1, wherein the method upregulates an apoptosis gene signature in the cells of the LMs or inhibits proliferation of the cells of the LMs.

10. The method of claim 1, wherein treating LMs in a subject in need thereof comprises reducing the likelihood of recurrence of LMs in the subject.

11. A method of killing or inhibiting proliferation a tumor cell comprising a mutation in mediator complex subunit 12 (MED12), the method comprising contacting the tumor cell with an effective amount of a signal transducer and activator of transcription 3 (STAT3) pathway inhibitor to kill or inhibit proliferation of the tumor cell.

12. The method of claim 11 wherein the STAT3 pathway inhibitor comprises a STAT3 specific inhibitor or a Janus kinase (JAK) inhibitor.

13. The method of claim 12, wherein the STAT3 specific inhibitor is S3I-201, C188-9, WP1066, or VVD-130850.

14. The method of claim 12, wherein the JAK inhibitor is selected from abrocitinib, fedratinib, deucravacitinib, delgocitinib, baricitinib, upadacitinib, ruxolitinib, tofacitinib, and filgotinib.

15. The method of claim 11, wherein the tumor cell is a uterine leiomyoma (LM) cell.

16. The method of claim 11, wherein the mutation in MED12 comprises a mutation that alters MED12 function and inactivates CDK8/19 kinase activity.

17. The method of claim 16, wherein the mutation in MED12 comprises a G44 mutation, with reference to SEQ ID NO: 1.

18. The method of claim 17, wherein the G44 mutation is a G44D substitution mutation.

19. The method of claim 11, wherein the method upregulates an apoptosis gene signature in the cells of the LMs.

20. A method of reducing recurrence likelihood of uterine leiomyomas (LMs) in a subject in need thereof, the method comprising administering a therapeutically effective amount of a signal transducer and activator of transcription 3 (STAT3) pathway inhibitor to the subject to reduce the recurrence likelihood of the uterine leiomyomas in the subject.
